SEQUENCE LISTING (21 sequences)

SEQ ID NO: 1 - protein, 559 aa, Pseudomonas sp.
MSNKSNDELKYQASENTLGLNPVVGLRGKDLLASARMVLRQAIKQPVHSVKHVAHFGLELKNVLLGKSGLQPTSDDRRFADPAWSQNPLYKRYLQTYLAWRKELHDWIDESNLAPKDVARGHFVINLMTDAMAPTNTAANPAAVKRFFETGGKSLLDGLSHLAKDLVHNGGMPSQVNMGAFEVGKSLGVTEGAVVFRNDVLELIQYKPTTEQVYERPLLVVPPQINKFYVFDLSPDKSLARFCLRNNVQTFIVSWRNPTKEQREWGLSTYIEALKEAVDVVTAITGSKDVNMLGACSGGITCTALLGHYAAIGENKVNALTLLVTVLDTTLDSDVALFVNEQTLEAAKRHSYQAGVLEGRDMAKVFAWMRPNDLIWNYWVNNYLLGNEPPVFDILFWNNDTTRLPAAFHGDLVELFKNNPLIRPNALEVCGTPIDLKQVTADIFSLAGTNDHITPWKSCYKSAQLFGGNVEFVLSSFGHIKSILNPPGNPKSRYMTSTEVAENADEWQANATKHTDSWWLHWQAWQAQRSGELKKSPTKLGSKAYPAGEAAPGTYVHER

SEQ ID NO: 2 - protein, 559 aa, unidentified
MSNKNSDDLNRQASENTLGLNPVIGLRGKDLLTSARMVLTQAIKQPIHSVKHVAHFGIELKNVMFGKSKLQPESDDRRFNDPAWSQNPLYKRYLQTYLAWRKELHDWIGNSKLSEQDINRAHFVITLMTDAMAPTNSAANPAAVKRFFETGGKSLLDGLTHLAKDLVNNGGMPSQVDMGAFEVGKSLGTTEGAVVFRNDVLELIQYRPTTEQVHERPLLVVPPQINKFYVFDLSPDKSLARFCLSNNQQTFIVSWRNPTKAQREWGLSTYIDALKEAVDVVSAITGSKDINMLGACSGGITCTALLGHYAALGEKKVNALTLLVSVLDTTLDSQVALFVDEKTLEAAKRHSYQAGVLEGRDMAKVFAWMRPNDLIWNYWVNNYLLGNEPPVFDILFWNNDTTRLPAAFHGDLIEMFKNNPLVRANALEVSGTPIDLKQVTADIYSLAGTNDHITPWKSCYKSAQLFGGKVEFVLSSFGHIKSILNPPGNPKSRYMTSTDMPATANEWQENSTKHTDSWWLHWQAWQAERSGKLKKSPTSLGNKAYPSGEAAPGTYVHER

SEQ ID NO: 3 - protein, 560 aa, Pseudomonas sp.
MREKPTPGLLPTPATFINAQSAITGLRGRDLFSTLRSVAAHGLRHPVRSARHVLALGGQLGRVLLGETLHTPNPKDNRFADPTWRLNPFYRRSLQAYLSWQKQVKSWIDESGMSDDDRARAHFVFALLNDAVSPSNTLLNPLAIKELFNSGGNSLVRGLSHLFDDLMHNNGLPSQVTKHAFEIGKTVATTAGSVVFRNELLELMQYKPMSEKQYAKPLLIVPPQINKYYIFDLSPGNSFVQYALKNGLQVFVVSWRNPDVRHREWGLSSYVEALEEALNVCRAITGARDVNLMGACAGGLTIAALQGHLQAKRQLRRVSSASYLVSLLDSQIDSPATLFADEQTLEAAKRHSYQRGVLEGRDMAKIFAWMRPNDLIWNYWVNNYLLGKEPPAFDILYWNSDNTRLPAAFHGDLLDFFKHNPLTHPGGLEVCGTPIDLQKVNVDSFSVAGINDHITPWDAVYRSTLLLGGDRRFVLSNSGHIQSILNPPSNPKSNYIENPKLSGDPRAWYYDGTHVEGSWWPRWLSWIQERSGTQRETLMALGNQNYPPMEAAPGTYVRVR

SEQ ID NO: 4 - protein, 559 aa, Pseudomonas aeruginosa
MSQKNNNELPKQAAENTLNLNPVIGIRGKDLLTSARMVLLQAVRQPLHSARHVAHFSLELKNVLLGQSELRPGDDDRRFSDPAWSQNPLYKRYMQTYLAWRKELHSWISHSDLSPQDISRGQFVINLLTEAMSPTNSLSNPAAVKRFFETGGKSLLDGLGHLAKDLVNNGGMPSQVDMDAFEVGKNLATTEGAVVFRNDVLELIQYRPITESVHERPLLVVPPQINKFYVFDLSPDKSLARFCLRNGVQTFIVSWRNPTKSQREWGLTTYIEALKEAIEVVLSITGSKDLNLLGACSGGITTATLVGHYVASGEKKVNAFTQLVSVLDFELNTQVALFADEKTLEAAKRRSYQSGVLEGKDMAKVFAWMRPNDLIWNYWVNNYLLGNQPPAFDILYWNNDTTRLPAALHGEFVELFKSNPLNRPGALEVSGTPIDLKQVTCDFYCVAGLNDHITPWESCYKSARLLGGKCEFILSNSGHIQSILNPPGNPKARFMTNPELPAEPKAWLEQAGKHADSWWLHWQQWLAERSGKTRKAPASLGNKTYPAGEAAPGTYVHER

SEQ ID NO: 5 - protein, 560 aa, Pseudomonas aeruginosa
MREKQESGSVPVPAEFMSAQSAIVGLRGKDLLTTVRSLAVHGLRQPLHSARHLVAFGGQLGKVLLGDTLHQPNPQDARFQDPSWRLNPFYRRTLQAYLAWQKQLLAWIDESNLDCDDRARARFLVALLSDAVAPSNSLINPLALKELFNTGGISLLNGVRHLLEDLVHNGGMPSQVNKTAFEIGRNLATTQGAVVFRNEVLELIQYKPLGERQYAKPLLIVPPQINKYYIFDLSPEKSFVQYALKNNLQVFVISWRNPDAQHREWGLSTYVEALDQAIEVSREITGSRSVNLAGACAGGLTVAALLGHLQVRRQLRKVSSVTYLVSLLDSQMESPAMLFADEQTLESSKRRSYQHGVLDGRDMAKVFAWMRPNDLIWNYWVNNYLLGRQPPAFDILYWNNDNTRLPAAFHGELLDLFKHNPLTRPGALEVSGTAVDLGKVAIDSFHVAGITDHITPWDAVYRSALLLGGQRRFILSNSGHIQSILNPPGNPKACYFENDKLSSDPRAWYYDAKREEGSWWPVWLGWLQERSGELGNPDFNLGSAAHPPLEAAPGTYVHIR

SEQ ID NO: 6 - protein, 559 aa, Pseudomonas oleovorans
MSNKNNDELQRQASENTLGLNPVIGIRRKDLLSSARTVLRQAVRQPLHSAKHVAHFGLELKNVLLGKSSLAPESDDRRFNDPAWSNNPLYRRYLQTYLAWRKELQDWIGNSDLSPQDISRGQFVINLMTEAMAPTNTLSNPAAVKRFFETGGKSLLDGLSNLAKDLVNNGGMPSQVNMDAFEVGKNLGTSEGAVVYRNDVLELIQYKPITEQVHARPLLVVPPQINKFYVFDLSPEKSLARYCLRSQQQTFIISWRNPTKAQREWGLSTYIDALKEAVDAVLAITGSKDLNMLGACSGGITCTALVGHYAALGENKVNALTLLVSVLDTTMDNQVALFVDEQTLEAAKRHSYQAGVLEGSEMAKVFAWMRPNDLIWNYWVNNYLLGNEPPVFDILFWNNDTTRLPAAFHGDLIEMFKSNPLTRPDALEVCGTPIDLKQVKCDIYSLAGTNDHITPWQSCYRSAHLFGGKIEFVLSNSGHIQSILNPPGNPKARFMTGADRPGDPVAWQENATKHADSWWLHWQSWLGERAGELEKAPTRLGNRAYAAGEASPGTYVHER

SEQ ID NO: 7 - protein, 559 aa, unidentified
MSNKNNDELQRQASENTLGLNPVIGIRRKDLLSSARTVLRQAVRQPLHSAKHVAHFGLELKNVLLGKSSLAPESDDRRFNDPAWSNNPLYRRYLQTYLAWRKELQDWIGNSDLSPQDISRGQFVINLMTDAMAPTNTLSNPAAVKRFFETGGKSLLDGLSNLAKDLVNNGGMPSQVNMDAFEVGKNLGTSEGAVVYRNDVLELIQYKPITEQVHARPLLVVPPQINKFYVFDLSPEKSLARYCLRSQQQTFIISWRNPTKAQREWGLSTYIDALKEAVDAVLAITGSKDLNMLGACSGGITCTALVGHYAALGENKVNALTLLVSVLDTTMDNQVALFVDEQTLEAAKRHSYQAGVLEGSEMAKVFAWMRPNDLIWNYWVNNYLLGNEPPVFDILFWNNDTTRLPAAFHGDLIEMFKSNPLTRPDALEVCGTPIDLKQVKCDIYSLAGTNDHITPWQSCYRSAHLFGGKIEFVLSNFGHIKSILNPPGNPKARFMTGADRPGDPVAWQENATKHADSWWLHWQSWLGERAGELEKAPTRLGNRAYAAGEASPGTYVHER

SEQ ID NO: 8 - protein, 560 aa, Pseudomonas oleovorans
MKDKPAKGTPTLPATSMNVQNAILGLRGRDLISTLRNVSRQSLRHPLHTAHHLLALGGQLGRVILGDTPLQPNPRDPRFSDPTWSQNPFYRRGLQAYLAWQKQTRLWIEESHLDDDDRARAHFLFNLINDALAPSNSLLNPLAVKELFNSGGQSLVRGVAHLLDDLRHNDGLPRQVDERAFEVGGNLAATAGAVVFRNELLELIQYKPMSEKQHARPLLVVPPQINKFYIFDLSSTNSFVQYMLKNGLQVFMVSWRNPDPRHREWGLSSYVQALEEALNACRSISGNRDPNLMGACAGGLTMAALQGHLQAKHQLRRVRSATYLVSLLDSKFESPASLFADEQTIEAAKRRSYQRGVLDGAEVARIFAWMRPNDLIWNYWVNNYLLGKTPPAFDILYWNADSTRLPAALHGDLLDFFKLNPLTHPAGLEVCGTPIDLQKVELDSFTVAGSNDHITPWDAVYRSALLLGGDRRFVLANSGHIQSIINPPGNPKAYYLANPKLSSDPRAWLHDAKRSEGSWWPLWLEWITARSGPLKAPRSELGNATYPPLGPAPGTYVLTR

SEQ ID NO: 9 - protein, 560 aa, unidentified
MKDKPAKGTPTLPATSMNVQNAILGLRGRDLISTLRNVSRQSLRHPLHTAHHLLALGGQLGRVILGDTPLQPNPRDPRFSDPTWSQNPFYRRGLQAYLAWQKQTRLWIEESHLDDDDRARAHFLFNLINDALAPSNSLLNPLAVKELFNSGGQSLVRGVAHLLDDLRHNDGLPRQVDERAFEVGGNLAATAGAVVFRNELLELIQYKPMSEKQHARPLLVVPPQINKFYIFDLSSTNSFVQYMLKNGLQVFMVSWRNPDPRHREWGLSSYVQALEEALNACRSISGNRDPNLMGACAGGLTMAALQGHLQAKHQLRRVRSATYLVSLLDSKFESPASLFADEQTIEAAKRRSYQRGVLDGAEVARIFAWMRPNDLIWNYWVNNYLLGKTPPAFDILYWNADSTRLPAALHGDLLDFFKLNPLTHPAGLEVCGTPIDLQKVELDSFTVAGSNDHITPWDAVYRSALLLGGDRRFVLANFGHIKSIINPPGNPKAYYLANPKLSSDPRAWLHDAKRSEGSWWPLWLEWITARSGPLKAPRSELGNATYPPLGPAPGTYVLTR

SEQ ID NO: 10 - protein, 559 aa, Pseudomonas putida
MSNKNNDELQRQASENTLGLNPVIGIRRKDLLSSARTVLRQAVRQPLHSAKHVAHFGLELKNVLLGKSSLAPDSDDRRFNDPAWSNNPLYRRYLQTYLAWRKELQDWVSSSDLSPQDISRGQFVINLMTEAMAPTNTLSNPAAVKRFFETGGKSLLDGLSNLAKDMVNNGGMPSQVNMDAFEVGKNLGTSEGAVVYRNDVLELIQYSPITEQVHARPLLVVPPQINKFYVFDLSPEKSLARFCLRSQQQTFIISWRNPTKAQREWGLSTYIDALKEAVDAVLSITGSKDLNMLGACSGGITCTALVGHYAAIGENKVNALTLLVSVLDTTMDNQVALFVDEQTLEAAKRHSYQAGVLEGSEMAKVFAWMRPNDLIWNYWVNNYLLGNEPPVFDILFWNNDTTRLPAAFHGDLIEMFKSNPLTRPDALEVCGTAIDLKQVKCDIYSLAGTNDHITPWPSCYRSAHLFGGKIEFVLSNSGHIQSILNPPGNPKARFMTGADRPGDPVAWQENATKHADSWWLHWQSWLGERAGALKKAPTRLGNRAYAAGEASPGTYVHER

SEQ ID NO: 11 - protein, 560 aa, Pseudomonas putida
MKDKPAKGSTTLPATRMNVQNAILGLRGRDLLSTLRNVGRHGLRHPLHTAHHLLALGGQLGRVMLGDTPYQPNPRDARFSDPTWSQNPFYRRGLQAYLAWQKQTRQWIDESHLNDDDRARAHFLFNLINDALAPSNSLLNPLAVKELFNTGGQSLVRGVAHLLDDLRHNDGLPRQVDERAFEVGANLAATPGAVVFRNELLELIQYSPMSEKQHARPLLVVPPQINKFYIFDLSATNSFVQYMLKSGLQVFMVSWRNPDPRHREWGLSSYVQALEEALNACRSISGNRDPNLMGACAGGLTMAALQGHLEAKQQLRRVRSATYLVSLLDSKFESPASLFADEQTIEAAKRRSYQRGVLDGGEVARIFAWMRPNDLIWNYWVNNYLLGKTPPAFDILYWNADSTRLPAALHGDLLEFFKLNPLTYASGLEVCGTPIDLQQVNIDSFTVAGSNDHITPWDAVYRSALLLGGERRFVLANSGHIQSIINPPGNPKAYYLANPKLSSDPRAWFHDAKRSEGSWWPLWLEWITARSGLLKAPRTELGNATYPPLGPAPGTYVLTR

SEQ ID NO: 12 - protein, 583 aa, Pseudomonas putida
MTEKKNGNNSSTIAPALDMQAHVAWAQAWSSISPESSLLAWTDWASHLANSPGKQAELLAFAGSLSEQWMSLLKKSLVSPDQEVTPPEPSPAYDRRFNDPAWDQWPYNLYRSSFLIQSKWWEQATQGVWGVDPQHERLLAFGAKQWLEIVSPTNSALFNPVVLRKTIEEQGANLARGMSNFLDDLRRQLSGEPPAGTENFVVGRDVAVTEGKVVLRNQLIELIQYTPTTEKVHPEPILIIPAWIMKYYVLDLSPHNSLIRYLVAQGHTVFCISWRNPDAEDRDLGMDEYLEFGLHAALDAVTSIVPNHGIHAAGYCLGGTLLAIGASAMARDGDTRLVSVSLLAAQTDFSEPGELGLFINQSQVALLEASMAQTGYLSSSQMSGVFQLLRAYDLIWSRMIDEYVLGDRRPMTDLMAWNADGTRLPAKMHSQYLRRLYLNNDLSAGRYPVMGRPVSVGDITVPMFCVGTASDHIAPWRSVYKLHLLTSAELTFVLTTGGHNGGIVSEPGRGKRQYQIHTRAVNEGYMAPDQWQATAQTHPDSWWQAWSAWLQERSGDVVAPPLMGAESNGYPAICDAPGEYVRS

SEQ ID NO: 13 - protein, 583 aa, unidentified
MTEKKNGNNSSTIAPALDMQAHVAWAQAWSSISPESSLLAWTDWASHLANSPGKQAELLAFAGSLSEQWMSLLKKSLVSPDQEVTPPEPSPAYDRRFNDPAWDQWPYNLYRSSFLIQSKWWEQATQGVWGVDPQHERLLAFGAKQWLDIVSPTNSALFNPVVLRKTIEEQGANLARGMSNFLDDLRRQLSGEPPAGTENFVVGRDVAVTEGKVVLRNQLIELIQYTPTTEKVHPEPILIIPAWIMKYYVLDLSPHNSLIRYLVAQGHTVFCISWRNPDAEDRDLGMDEYLEFGLHAALDAVTSIVPNHGIHAAGYCLGGTLLAIGASAMARDGDTRLVSVSLLAAQTDFSEPGELGLFINQSQVALLEASMAQTGYLSSSQMSGVFQLLRAYDLIWSRMIDEYVLGDRRPMTDLMAWNADGTRLPAKMHSQYLRRLYLNNDLSAGRYPVMGRPVSVGDITVPMFCVGTASDHIAPWRSVYKLHLLTSAELTFVLTTGGHNGGIVSEPGRGKRQYQIHTRAVNEGYMAPDQWQATAQTHPDSWWQAWSAWLQERSGDVVAPPLMGAESNGYPAICDAPGEYVRS

SEQ ID NO: 14 - protein, 399 aa, Peptoclostridium difficile
MLLEGVKVVELSSFIAAPCCAKMLGDWGAEVIKIEPIEGDGIRVMGGTFKSPASDDENPMFELENGNKKGVSINVKSKEGVEILHKLLSEADIFVTNVRVQALEKMGIAYDQIKDKYPGLIFSQILGYGEKGPLKDKPGFDYTAYFARGGVSQSVMEKGTSPANTAAGFGDHYAGLALAAGSLAALHKKAQTGKGERVTVSLFHTAIYGMGTMITTAQYGNEMPLSRENPNSPLMTTYKCKDGRWIQLALIQYNKWLGKFCKVINREYILEDDRYNNIDSMVNHVEDLVKIVGEAMLEKTLDEWSALLEEADLPFEKIQSCEDLLDDEQAWANDFLFKKTYDSGNTGVLVNTPVMFRNEGIKEYTPAPKVGQHTVEVLKSLGYDEEKINNFKDSKVVRY

SEQ ID NO: 15 - protein, 524 aa, Anaerotignum propionicum
MRKVPIITADEAAKLIKDGDTVTTSGFVGNAIPEALDRAVEKRFLETGEPKNITYVYCGSQGNRDGRGAEHFAHEGLLKRYIAGHWATVPALGKMAMENKMEAYNVSQGALCHLFRDIASHKPGVFTKVGIGTFIDPRNGGGKVNDITKEDIVELVEIKGQEYLFYPAFPIHVALIRGTYADESGNITFEKEVAPLEGTSVCQAVKNSGGIVVVQVERVVKAGTLDPRHVKVPGIYVDYVVVADPEDHQQSLDCEYDPALSGEHRRPEVVGEPLPLSAKKVIGRRGAIELEKDVAVNLGVGAPEYVASVADEEGIVDFMTLTAESGAIGGVPAGGVRFGASYNADALIDQGYQFDYYDGGGLDLCYLGLAECDEKGNINVSRFGPRIAGCGGFINITQNTPKVFFCGTFTAGGLKVKIEDGKVIIVQEGKQKKFLKAVEQITFNGDVALANKQQVTYITERCVFLLKEDGLHLSEIAPGIDLQTQILDVMDFAPIIDRDANGQIKLMDAALFAEGLMGLKEMKS

SEQ ID NO: 16 - protein, 410 aa, Paeniclostridium sordellii
MDNRALLKGVRVVELSSFVAAPCCAKLLADWGAEVIKIEPLGGDGIRVMGGTFKSPCTDDENPMFELENGNKKGISVNVKTKEGVEILHKLLSKSDIFVTNVREKALAKMGLTYDQLKDDFPGLIHAHILGYGEEGPLKDKPGFDYTAYFARGGVSQSLMEKGTSPCNTAAGFGDHYAGISLTAGILAALYKKQITGEGDRVTVSLFHTALYGMGMMITTSQYGNEMPISRTEPNSPLMTTYKCKDGKWIQLALIQYNKWLPKFCEVINRPEIMKDDRFNDIKVMPMHVDEMVKIVEKAMLEKTLDEWSALLEEADLPFEKVQSCEDIINDDQVWANDFLFKTTYENGNEGVLVNGPVKFKTMGIKEYEPAPRLGQHTEEVLKSIGYTEEEILDMVNSQAIKLDDAKELV

SEQ ID NO: 17 - protein, 404 aa, Clostridium sp.
MDKNGLALEGIKIVELSSFVAAPSCAKVLADWGAEVIKVEPVQGDNLRIVGPVYNAPAKDEENPMFELENGNKMGIAINTGSEKGKEVLGKLLQDADVFITNVREKALERSGLSYEQLKDKYPGLIHAHILGYGEKGPLKDKPGFDYTAYFARGAVSISLMEKGTSPANTNAGFGDHYAGMSLAAGILAALHKKTQTGKGDRVTVSLYHTAIFGMGLMITTAQYGNKMPLSRRTPNNPLATTFKCKDDRWIQLALLSYDKWFPKFCKEVINRLDLIEDERFNTQDEVVKHVETFVGILEQEMIKKTLGEWAELLDKADLPYEKLQTCEDILEDEQAWANDYLFKKTYDNGNTGVLVNTPVKFNESGIKPYKPSPKLGEDTEEILLGLGYSKEEIEEMRKGKAIR

SEQ ID NO: 18 - protein, 404 aa, Clostridium botulinum
MTKEGLALEGVKVVELSSFVAAPSCSKLLADWGADVIKIEPIQGDNIRVVGGVYNSPARDDENPMFELENGNKRGIAINTRSEKGKEVLGKLLKDADVFVTNVREKALQRSGLSYDQLKDKYPSLIHAHILGYGEKGPLKDKPGFDYTAYFARGAVSTSLMEKGTSPANTNAGFGDHYAGMSLAAGILAALHRKTLTGKGDRVTVSLYHTAIFGMGLMITTAQYGNKMPLSRRTPNNPLATTYRCKDDRWIQLALLKYDAWFPKFCKEVINRPDLIEDSRFNKQSEVVKHVETFVGILEGEFIKKDLKEWADLLDKADLPYEKLQYCEDILEDEQAWANDYLFKTTYDSGNTGVLVNSPVKFSEAGMRPYKAAPKIGEDTEVVLTSLGYSKEEIEEMRKEESIK

SEQ ID NO: 19 - protein, 402 aa, Marinisporobacter balticus
MSDKWLLKGVKVVEFATFVAAPSCAKMLADWGADVIKVEPISGEGQRTVGLAYSSPATEDENPWFENENFNKKSICINVKSAEGKEAFHKLISQADVFVTNVRVGALKKIGLSYEQLKEQHPGLVFAQILGYGEKGPLKDKPGFDYTSYFARGGVMASLMEKDTSPLNGAAGFGDHYSGIALAAGTCAALVNKARTGKGEKVTVSLYHMGIYGLGCMIFSDQYGNKMPMTRLSPNSPVCNSYQCKDGRWIQLALIQYDQWIGRFFKAIKREELINDDRYNTRTGMVQHVEEMVSMVAEAMLEKTLDEWEETLLEYDVPFERVQRCEDIVKDEQAWANDYLVKKTYDSGNEGILINTPVKFGEMGIREMTPAPRITENTDEILTAIGYSNEKIEEMKEIKAVR

SEQ ID NO: 20 - protein, 403 aa, Clostridium tagluense
MDDNKWLLKGIKVVEFATFIAAPCAARMLADWGADVIKVEPISGENMRGIGSVYSSPCQEDENPWFENENFNKKSICVNVKSTEGMEVFHKLLEKADIFVTNVRVQALAKLGLSYEQLKEKYPGLIFVQALGYGEEGPLKDKPGFDYTSYFARGGVMSSLMEKGTTPTNVAAGFGDHYAGIALAAGACAALVKKAKTGTGEKITVSLYHMGIYGLGSMIMSDQYGNKMPMSRLTPNSPVCNSYQCKDEKWIQLALIQYDQWIERFFNAINREDLMNDDRYNTRNGMVENVESMVTIVAEAMLKKTLAQWEKVLMECDIPFERVQSCADIAVDEQAWANDYLVKKTYDSGNEGILVNSPVKFGEMGIREMTPAPRLEENTDEILSSIGYNMEEIQTLKSGKLVR

SEQ ID NO: 21 - protein, 524 aa, Clostridium propionicum; identical to SEQ ID NO: 15

(19) United States
(12) Patent Application Publication
(10) Pub. No.: US 2024/0384311 A1
(43) Pub. Date: Nov. 21, 2024

(54) POLYHYDROXYALKANOATES AND METHODS OF MAKING THEREOF

(71) Applicant: The Board of Trustees of the Leland Stanford Junior University, Stanford, CA (US)

(72) Inventors: Nils Averesch, Essen (DE); Vince Pane, Stanford, CA (US); Robert Waymouth, Palo Alto, CA (US); Craig Criddle, San Luis Obispo, CA (US)

(21) Appl. No.: 18/570,566

(22) PCT Filed: Jul. 7, 2022
(86) PCT No.: PCT/US2022/073521

§ 371 Date: Dec. 14, 2023

Related U.S. Application Data
(60) Provisional application No. 63/220,165, filed on Jul. 9, 2021.

(51) Int. Cl. C12P 7/625; C12N 1/20; C12N 9/10; C12N 9/14; C12N 15/52; C12R 1/01

(52) U.S. Cl. CPC C12P 7/625; C12N 1/20; C12N 9/1029; C12N 9/13; C12N 9/14; C12N 15/52; C12R 2001/01; C12Y 203/01; C12Y 208/03001

(57) ABSTRACT

Provided are microorganisms for making polyhydroxylalkanoate (PHA) compounds. For instance, the microorganism can include a polyhydroxylalkanoate (PHA) synthase (phaC) gene and one or both of an isocaprenoyl-CoA:2-hydroxyisocaproate CoA-transferase (hadA) gene and a propionate CoA-transferase (pct) gene. In some cases, the species of the microorganism is a Cupriavidus necator bacteria that has been genetically modified to include the PHA and hadA or pct genes.

### CROSS-REFERENCE TO RELATED APPLICATIONS

This application claims the benefit of U.S. Provisional Patent Application No. 63/220,165 filed Jul. 9, 2021, which application is incorporated herein by reference in its entirety.

### GOVERNMENT RIGHTS

This invention was made with government support under grant number NNX17AJ31G awarded by the United States National Aeronautics and Space Administration. The government has certain rights in the invention.

### INCORPORATION BY REFERENCE OF A SEQUENCE LISTING PROVIDED AS A TEXT FILE

A Sequence Listing is provided herewith as a text file, “STAN-18700WO_SEQ_LIST.xml,” created on Jul. 7, 2022 and having a size of 29 kilobytes. The contents of the text file are incorporated by reference herein in their entirety.

### INTRODUCTION

Synthetic materials are integral components of consumables and durable goods and indispensable in the modern world. Polyesters are among the most versatile bulk-as well as specialty-polymers and their sustainable production, as well as fate at end-of-life are of great environmental concern. Polyhydroxyalkanoates (PHAs), a class of biological thermoplastic polyesters, are potential bio-degradable replacements for these materials. The most common natural bio-polyesters, poly (3-hydroxybutyrate), can be produced outgoing from non-edible carbon-sources such as carbon dioxide and methane.

However, commercial competitiveness with synthetic plastics and shortcomings of the materials properties, have so far hampered its success on global market scale. Allowing bio-production of advanced PHAs with superior properties could change this, especially materials that can directly replace industrial (petrochemical-based) polymers could be useful, to make PHAs not only economically viable, but commercially attractive, without the need for extensive modifications to the existing processing-and recycling-infrastructure.

In addition, the melting point and glass transition temperatures of plastics can be important for practical applications. The glass transition temperature relates to the temperature range over which a glass transition occurs, e.g., where the material transitions from a relatively hard and brittle state (e.g., a “glassy” state) to a more viscous and malleable state. Melting point refers to the temperature at which the material melts.

For practical applications, it can be desirable to first generate the plastic material and then shape it into a commercial product, such as a cup, fork, or spoon. Materials with lower melting points and glass transition temperatures commonly become more malleable at lower temperatures, making it easier to shape them into commercial products. However, the plastic can also begin to chemically decompose if exposed to a sufficiently high temperature. As such, materials with a sufficiently large difference between the glass transition or melting temperatures and the thermal decomposition temperature can be advantageous since they can be readily shaped into desired commercial products with minimal amounts of thermal decomposition.

### SUMMARY

Provided are microorganisms for making polyhydroxylalkanoate (PHA) compounds. For instance, the microorganism can include a polyhydroxylalkanoate (PHA) synthase (phaC) gene and one or more of an isocaprenoyl-CoA:2-hydroxyisocaproate CoA-transferase (hadA) gene, a propionate CoA-transferase (pct) gene. In some cases, the species of the microorganism is a Cupriavidus necator bacteria that has been genetically modified to contain the PHA and hadA or pct genes.

### BRIEF DESCRIPTION OF THE DRAWINGS

FIG. 1 shows relative yields due to different carbon sources.

FIG. 2 shows the first part of a proposed mechanism of polymer production.

FIG. 3 shows a second part of a proposed mechanism of polymer production.

FIG. 4 shows vials containing various produced polymers.

FIGS. 5A-5M show amino acid sequences of PHA synthases according to certain embodiments.

FIGS. 6A-6E show alignment of amino acid sequences of PHA synthases listed in FIGS. 5A-5M. SEQ:1 is SEQ ID NO:1, SEQ:2 is SEQ ID NO:2, SEQ:3 is SEQ ID NO:3, SEQ:4 is SEQ ID NO:4, SEQ:5 is SEQ ID NO:5, SEQ:6 is SEQ ID NO:6, SEQ:7 is SEQ ID NO:7, SEQ:8 is SEQ ID NO:8, SEQ:9 is SEQ ID NO:9, SEQ:10 is SEQ ID NO:10, SEQ:11 is SEQ ID NO:11, SEQ:12 is SEQ ID NO:12, and SEQ:13 is SEQ ID NO:13.

FIGS. 7A-7G show amino acid sequences of hydroxyl-CoA transferases according to certain embodiments.

### DETAILED DESCRIPTION

Provided are microorganisms for making polyhydroxylalkanoate (PHA) compounds. For instance, the microorganism can include a polyhydroxylalkanoate (PHA) synthase gene and one or more of a an isocaprenoyl-CoA:2-hydroxyisocaproate CoA-transferase (hadA) gene, a propionate CoA-transferase (pct540) gene. In some cases, the species of the microorganism is a Cupriavidus necator bacteria that has been genetically modified to contain the PHA and hadA or pct540 genes.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a droplet” includes a plurality of such droplets and reference to “the discrete entity” includes reference to one or more discrete entities, and so forth. It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### DEFINITIONS

“Alkyl” refers to monoradical, branched or linear, cyclic or non-cyclic, saturated hydrocarbon group. Exemplary alkyl groups include methyl, ethyl, n-propyl, isopropyl, n-butyl, isobutyl, t-butyl, octyl, decyl, cyclopentyl, and cyclohexyl. In some cases, the alkyl group comprises 1 to 24 carbon atoms, such as 1 to 18 carbon atoms or 1 to 12 carbon atoms. The term “lower alkyl” refers to an alkyl groups with 1 to 6 carbon atoms.

“Alkenyl” refers to a monoradical, branched or linear, cyclic or non-cyclic hydrocarbonyl group that comprises a carbon-carbon double bond. Exemplary alkenyl groups include ethenyl, n-propenyl, isopropenyl, n-butenyl, isobutenyl, octenyl, decenyl, tetradecenyl, hexadecenyl, eicosenyl, and tetracosenyl. In some cases, the alkenyl group comprises 1 to 24 carbon atoms, such as 1 to 18 carbon atoms or 1 to 12 carbon atoms. The term “lower alkenyl” refers to an alkyl groups with 1 to 6 carbon atoms.

“Alkynyl” refers to a monoradical, branched or linear, cyclic or non-cyclic hydrocarbonyl group that comprises a carbon-carbon triple bond. Exemplary alkynyl groups include ethynyl and n-propynyl. In some cases, the alkenyl group comprises 1 to 24 carbon atoms, such as 1 to 18 carbon atoms or 1 to 12 carbon atoms. The term “lower alkenyl” refers to an alkyl groups with 1 to 6 carbon atoms.

“Heterocyclyl” refers to a monoradical, cyclic group that contains a heteroatom (e.g., O, S, N) in as a ring atom and that is not aromatic (i.e., distinguishing heterocyclyl groups from heteroaryl groups). Exemplary heterocyclyl groups include piperidinyl, tetrahydroturanyl, dihydrofuranyl, and thiocanyl.

“Aryl” refers to an aromatic group containing at least one aromatic ring wherein each of the atoms in the ring are carbon atoms, i.e., none of the ring atoms are heteroatoms (e.g., O, S. N). In some cases, the aryl group has a second aromatic ring, e.g., that is fused to the first aromatic ring. Exemplary aryl groups are phenyl, naphthyl, biphenyl, diphenylether, diphenylamine, and benzophenone.

“Heteroaryl” refers to an aromatic group containing at least one aromatic ring wherein at least one of the atoms in the ring is a heteroatom (e.g., O, S, N). Exemplary heteroaryl groups include furyl, thiophenyl, imidazoyl, and pyrimidinyl.

The term “substituted” refers the removal of one or more hydrogens from an atom (e.g., from a C or N atom) and their replacement with a different group. For instance, a hydrogen atom on a phenyl (—C₆H₅) group can be replaced with a methyl group to form a —C₆H₄CH₃ group. Thus, the —C₆H₄CH₃ group can be considered a substituted aryl group. As another example, two hydrogen atoms from the second carbon of a propyl (—CH₂CH₂CH₃) group can be replaced with an oxygen atom to form a —CH₂C (O) CH₃ group, which can be considered a substituted alkyl group. However, replacement of a hydrogen atom on a propyl (—CH₂CH₂CH₃) group with a methyl group (e.g., giving —CH₂CH(CH₃)CH₃) is not considered a “substitution” as used herein since the starting group and the ending group are both alkyl groups. However, if the propyl group was substituted with a methoxy group. thereby giving a —CH₂CH(OCH₃)CH₃ group, the overall group can no longer be considered “alkyl”, and thus is “substituted alkyl”. Thus, in order to be considered a substituent, the replacement group is a different type than the original group. In addition, groups are presumed to be unsubstituted unless described as substituted. For instance, the term “alkyl” and “unsubstituted alkyl” are used interchangeably herein.

Exemplary substituents include alkoxy, substituted alkoxy, cycloalkyl, substituted cycloalkyl, cycloalkenyl, substituted cycloalkenyl, acyl, acylamino, acyloxy, amino, aminoacyl. aminoacyloxy, oxyaminoacyl, azido, cyano, alkyl, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR′R″, wherein R′ and R″ may be the same or different and are chosen from hydrogen. optionally substituted alkyl, cycloalkyl, alkenyl, cycloalkenyl, alkynyl, aryl, heteroaryl and heterocyclic.

Diradical groups are also described herein, i.e., in contrast to the monoradical groups such as alkyl and aryl described above. The term “alkylene” refers to the diradical version of an alkyl group, i.e., an alkylene group is a diradical, branched or linear, cyclic or non-cyclic, saturated hydrocarbon group. Exemplary alkylene groups include diylmethane (—CH₂—, which is also known as a methylene group), 1,2-diylethane (—CH₂CH₂—), and 1,1-diylethane (i.e., a CHCH₃ fragment where the first atom has two single bonds to other two different groups). The term “arylene” refers to the diradical version of an aryl group, e.g., 1,4-diylbenzene refers to a C₆H₄ fragment wherein two hydrogens that are located para to one another are removed and replaced with single bonds to other groups. The terms “alkenylene”, “alkynylene”, “heteroarylene”, and “heterocyclene” are also used herein.

“Acyl” refers to a group of formula —C(O)R wherein R is alkyl, alkenyl, or alkynyl. For example, the acetyl group has formula —C(O)CH₃.

“Alkoxy” refers to a group of formula —O(alkyl). Similar groups can be derived from alkenyl, alkynyl, and aryl groups as well.

“Amino” refers to the group —NRR′ wherein R and R′ are independently hydrogen or nonhydrogen substituents, with nonhydrogen substituents including, for example, alkyl, aryl, alkenyl, aralkyl, and substituted variants thereof.

“Halo” and “halogen” refer to the chloro, bromo, fluoro, and iodo groups.

“Carboxyl”, “carboxy”, and “carboxylate” refer to the —CO₂H group and salts thereof.

“Sulfonyl” refers to the group —SO₂R, wherein R is alkyl, alkenyl, alkynyl, aryl, heteroaryl, heterocyclyl, and substituted versions thereof. Exemplary sulfonyl groups includes —SO: CH₃ and —SO₂(C₆H₅).

Unless otherwise specified, reference to an atom is meant to include all isotopes of that atom. For example, reference to H is meant to include ¹H, ²H (i.e., D) and ³H (i.e., T), and reference to C is meant to include ¹²C and all isotopes of carbon (such as ¹³C). In addition, any groups described include all stereoisomers of that group.

### Microorganisms

Provided is genetically modified microorganism comprising:
a heterologous polyhydroxyalkanoate (PHA) synthase (phaC) gene; and
an isocaprenoyl-CoA:2-hydroxyisocaproate CoA-transferase (hadA) gene and/or a propionate CoA-transferase (pct) gene.

Provided is a genetically modified microorganism comprising:
a polyhydroxyalkanoate (PHA) synthase (phaC) gene; and
an isocaprenoyl-CoA:2-hydroxyisocaproate CoA-transferase (hadA) gene or a propionate CoA-transferase (pct) gene, or a combination thereof,
wherein the microorganism is capable of producing a PHA polymer.

In certain embodiments, the phaC gene is heterologous to the microorganism. In the certain embodiments, the phaC gene is a mutant PHA synthase (phaC1437) from Pseudomonas sp. MBEL 6-19. In the certain embodiments, the mutant phaC1437 encodes a mutant PHA synthase comprising the amino acid sequence:

MSNKSNDELKYQASENTLGLNPVVGLRGKDLLASARMVLRQAIKQPVHSVK HVAHFGLELKNVLLGKSGLQPTSDDRRFADPAWSQNPLYKRYLQTYLAWRKELHDWI DESNLAPKDVARGHFVINLMTDAMAPTNTAANPAAVKRFFETGGKSLLDGLSHLAKDL VHNGGMPSQVNMGAFEVGKSLGVTEGAVVFRNDVLELIQYKPTTEQVYERPLLVVPPQ INKFYVEDLSPDKSLARFCLRNNVQTFIVSWRNPTKEQREWGLSTYIEALKEAVDVVTA ITGSKDVNMLGACSGGITCTALLGHYAAIGENKVNALTLLVTVLDTTLDSDVALFVNE QTLEAAKRHSYQAGVLEGRDMAKVFAWMRPNDLIWNYWVNNYLLGNEPPVFDILFW NNDTTRLPAAFHGDLVELFKNNPLIRPNALEVCGTPIDLKQVTADIFSLAGTNDHITPWK SCYKSAQLFGGNVEFVLSSFGHIKSILNPPGNPKSRYMTSTEVAENADEWQANATKHTD SWWLHWQAWQAQRSGELKKSPTKLGSKAYPAGEAAPGTYVHER (SEQ ID NO:1).

The mutant PHA synthase comprises the following substitutions as compared to Pseudomonas sp. PHA synthase 1: E130D,S325T,S477F,Q481K, where residues are numbered with reference to SEQ ID NO:1.

In the certain embodiments, the phaC gene introduced into the microorganism encodes a PHA synthase that comprises an amino acid sequence having at least 80% identity (e.g., at least 85%, at least 90%, at least 93%, at least 95%, at least 96%, at least 97%, at least 98%, at least 99%, or a 100% identity) to the amino acid sequence of SEQ ID NO:1 and comprises 130D, 325T, 477F, and 481K, where residues are numbered with reference to SEQ ID NO:1.

A polynucleotide or polypeptide has a certain percent “sequence identity” to another polynucleotide or polypeptide, respectively, meaning that, when aligned, that percentage of bases or amino acids are the same, and in the same relative position, when comparing the two sequences. Sequence similarity can be determined in a number of different manners. Percent identity between a pair of sequences may be calculated by multiplying the number of matches in the pair by 100 and dividing by the length of the aligned region, including gaps. Identity scoring only counts perfect matches and does not consider the degree of similarity of amino acids to one another. Only internal gaps are included in the length, not gaps at the sequence ends.

Percent Identity=(matches×100)/length of aligned region (with gaps)

To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wisconsin, USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, California, USA. Of particular interest are alignment programs that permit gaps in the sequence. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences. See J. Mol. Biol. 48:443-453 (1970).

In certain embodiments, the PHA synthase may have conservative amino acid substitutions as compared to SEQ ID NO:1. The phrase “conservative amino acid substitution” refers to substitution of amino acid residues within the following groups: 1) L, I, M, V, F; 2) R, K; 3) F, Y, H, W, R; 4) G, A, T, S; 5) Q, N; and 6) D, E. Conservative amino acid substitutions may preserve the activity of the protein by replacing an amino acid(s) in the protein with an amino acid with a side chain of similar acidity, basicity, charge, polarity, or size of the side chain.

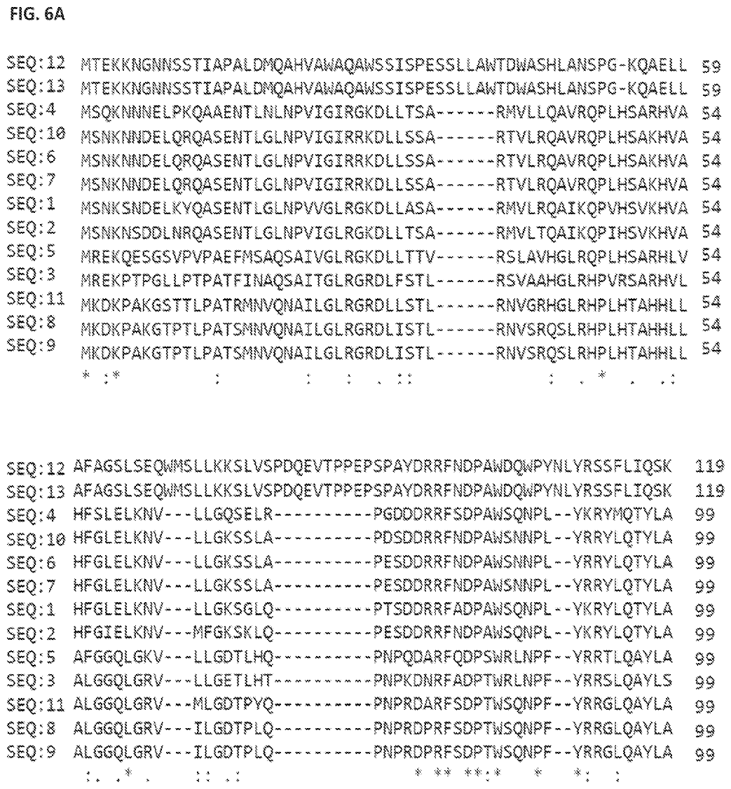

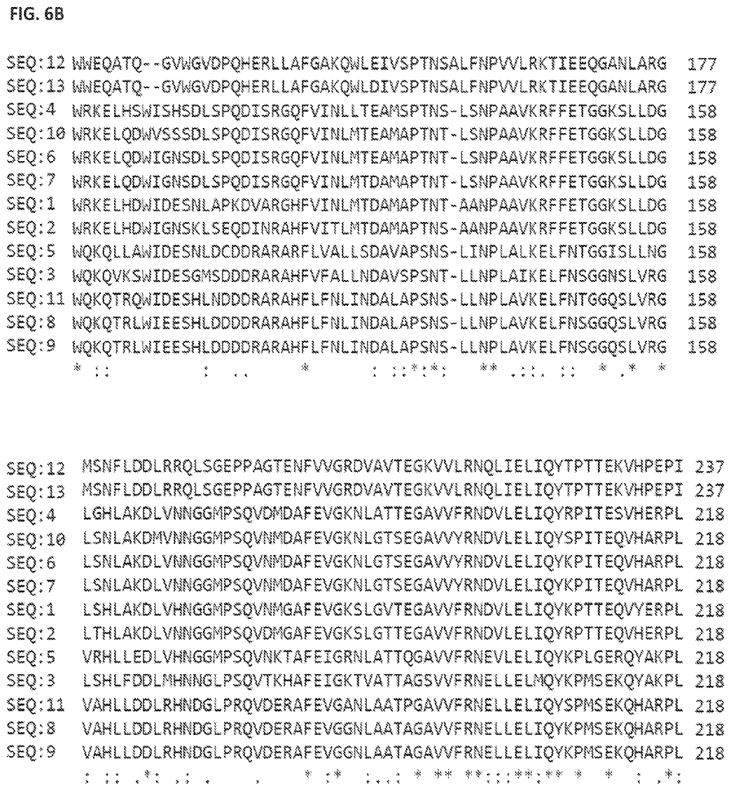

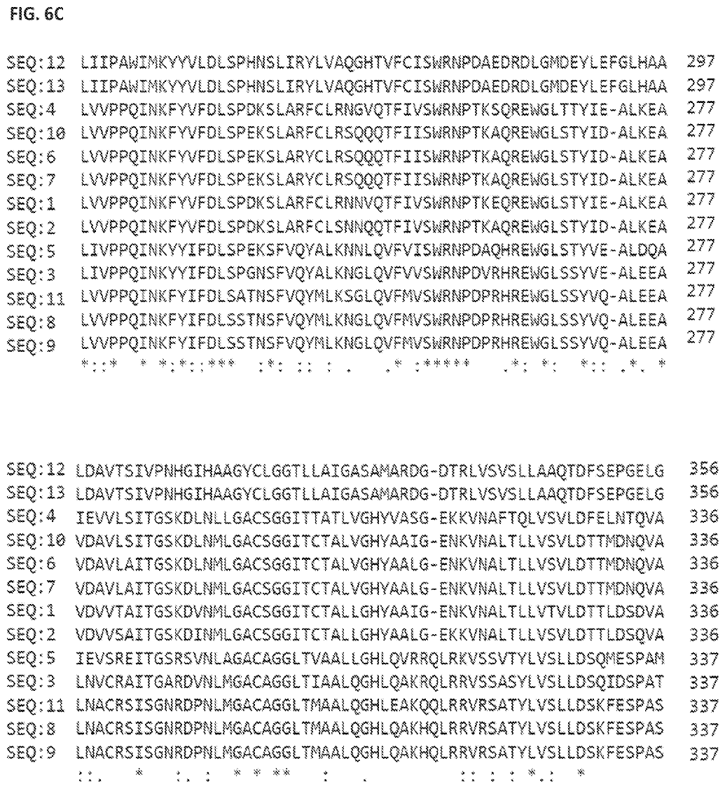

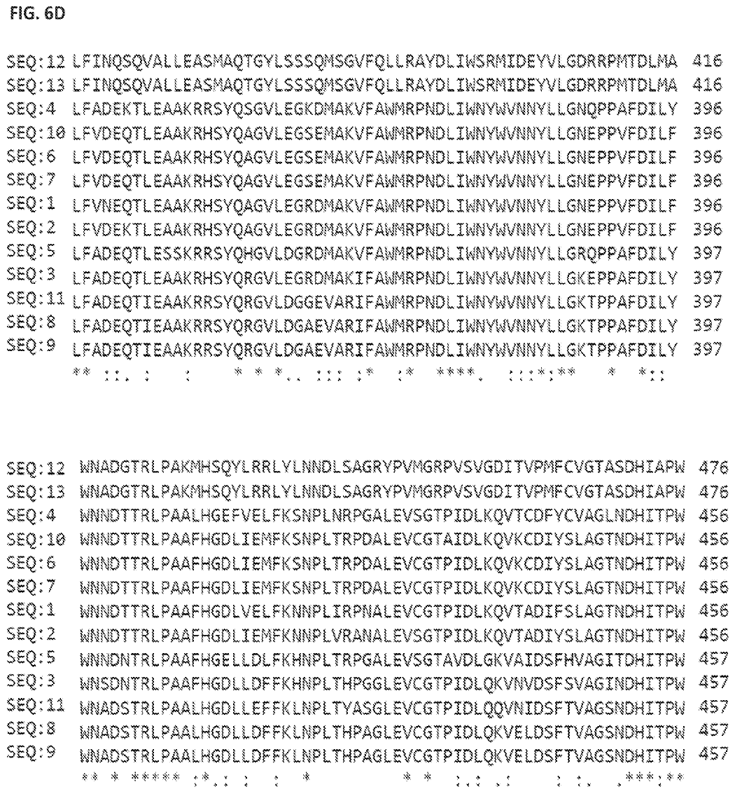

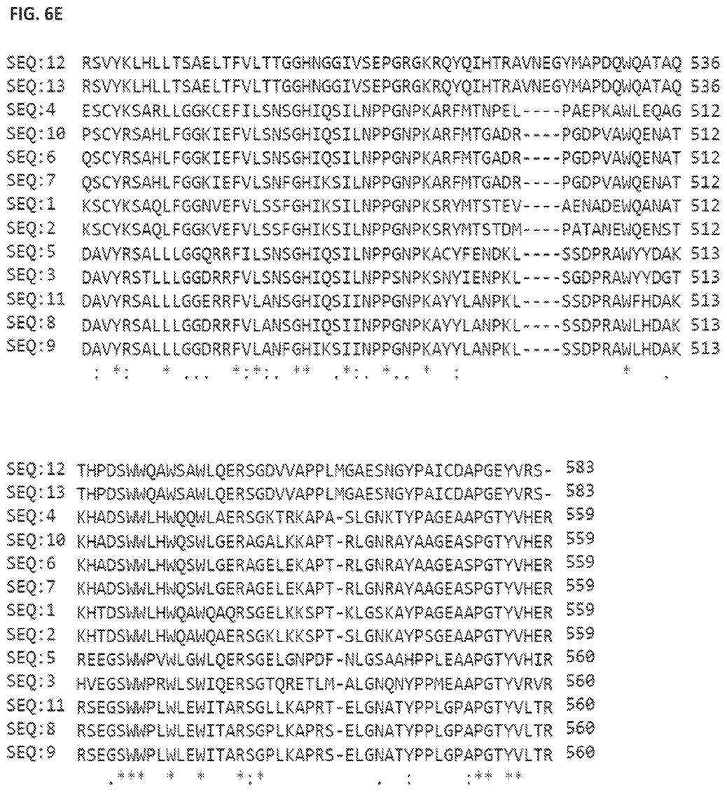

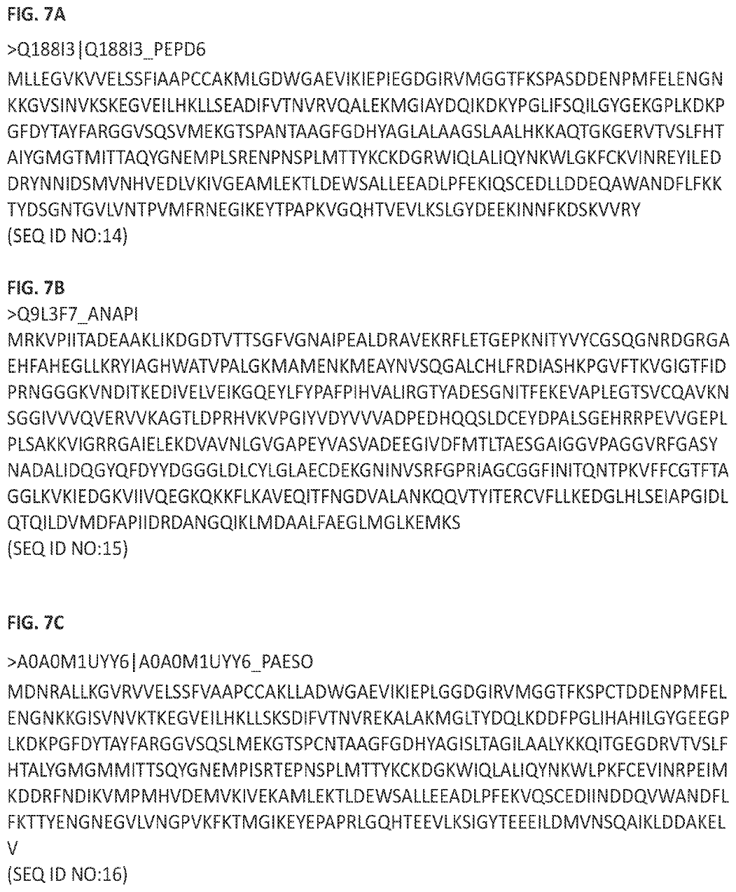

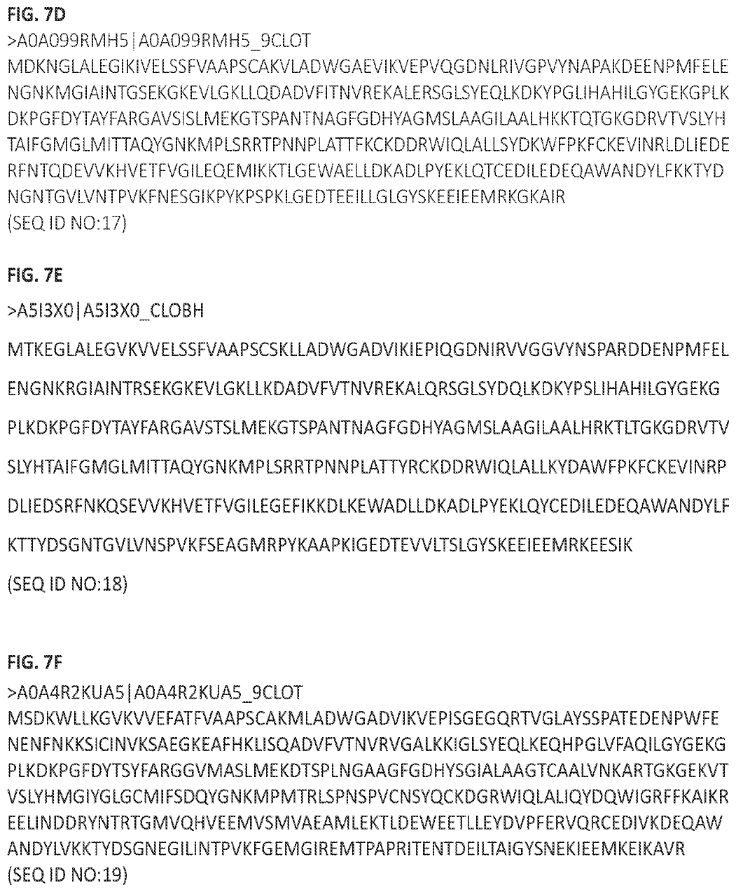

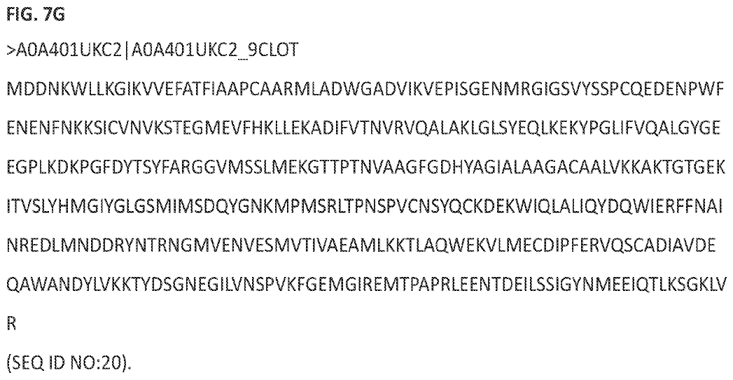

In certain embodiments, the PHA synthase may have an amino acid sequence that includes substitutions in regions not conserved between PHA synthase of different microorganisms. In certain embodiments, regions not conserved between different PHA synthases may be identified by conducting sequence alignments. See, for example, FIGS. 6A-6E.

In certain embodiments, the PHA synthase may have an amino acid sequence having at least 80% identity (e.g., at least 85%, at least 90%, at least 93%, at least 95%, at least 96%, at least 97%, at least 98%, at least 99%, or a 100% identity) to the amino acid sequence set forth in SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13 and comprises 130D, 325T, 477F, and 481K, where residues are numbered with reference to SEQ ID NO:1.

In certain embodiments, the microorganism is a Cupriavidus species bacterium. In certain embodiments, the microorganism is Cupriavidus necator (C. necator). In certain embodiments, the C. necator is genetically modified to not express an endogenous PHA synthase. In certain embodiments, the C. necator is genetically modified to delete the endogenous phaC gene. In certain embodiments, the C. necator is genetically modified to delete the endogenous phaC gene and to include a heterologous phaC gene, where the heterologous phaC gene encodes a PHA synthase that polymerizes various hydroxy carboxylates, including phloretic acid.

“Heterologous” in the context of recombinant cells, e.g., genetically modified microorganism, can refer to the presence of a nucleic acid (or gene product, such as a polypeptide) that is of a different genetic origin than the host cell in which it is present. For example, an amino acid or nucleic acid sequence from Pseudomonas species bacterium is heterologous to a Cupriavidus species bacterium.

In certain embodiments, the heterologous phaC gene is codon optimized for expression in the microorganism. In certain embodiments, the microorganism is a Cupriavidus species bacterium and the heterologous phaC gene is codon optimized for expression in the Cupriavidus species bacterium. In certain embodiments, the microorganism is C. necator. In certain embodiments, the C. necator is genetically modified to not express an endogenous PHA synthase. In certain embodiments, the microorganism is C. necator H16 ΔphaC1.

In certain embodiments, in addition to the genetic modification to express a heterologous PHA synthase, the microorganism is further genetically modified to express a hydroxyacyl-CoA-transferase. In certain embodiments, the microorganism is genetically modified to express a heterologous hydroxyacyl-CoA-transferase, where the hydroxyacyl-CoA-transferase is an isocaprenoyl-CoA:2-hydroxyisocaproate CoA-transferase encoded by a hadA gene derived from a Clostridium species bacterium. In certain embodiments, the hadA gene is derived from Clostridium species bacterium, C. difficile. In certain embodiments, the microorganism is encodes an isocaprenoyl-CoA:2-hydroxyisocaproate CoA-transferase comprising an amino acid sequence having at least 80% identity (e.g., at least 85%, at least 90%, at least 93%, at least 95%, at least 96%, at least 97%, at least 98%, at least 99%, or a 100% identity) to the amino acid sequence set forth in SEQ ID NO:14, SEQ ID NO:15, SEQ ID NO:16, SEQ ID NO:17, SEQ ID NO:18, SEQ ID NO:19, or SEQ ID NO:20.

In certain embodiments, the microorganism is genetically modified to express a heterologous hydroxyacyl-CoA-transferase, where the hydroxyacyl-CoA-transferase is a propionate CoA-transferase encoded by a pct gene derived from a Clostridium species bacterium. In certain embodiments, the pct gene is derived from Clostridium species bacterium, C. propionicum. In certain embodiments, the pct gene encodes a CoA-transferase comprising an amino acid sequence having at least 80% identity (e.g., at least 85%, at least 90%, at least 93%, at least 95%, at least 96%, at least 97%, at least 98%, at least 99%, or a 100% identity) to the amino acid sequence set forth in SEQ ID NO:21:

(SEQ ID NO: 21)
MRKVPIITADEAAKLIKDGDTVTTSGFVGNAIPEALDRAVEKRFL
ETGEPKNITYVYCGSQGNRDGRGAEHFAHEGLLKRYIAGHWATVP
ALGKMAMENKMEAYNVSQGALCHLFRDIASHKPGVFTKVGIGTFI
DPRNGGGKVNDITKEDIVELVEIKGQEYLFYPAFPIHVALIRGTY
ADESGNITFEKEVAPLEGTSVCQAVKNSGGIVVVQVERVVKAGTL
DPRHVKVPGIYVDYVVVADPEDHQQSLDCEYDPALSGEHRRPEVV
GEPLPLSAKKVIGRRGAIELEKDVAVNLGVGAPEYVASVADEEGI
VDFMTLTAESGAIGGVPAGGVRFGASYNADALIDQGYQFDYYDGG
GLDLCYLGLAECDEKGNINVSRFGPRIAGCGGFINITQNTPKVFF
CGTFTAGGLKVKIEDGKVIIVQEGKQKKFLKAVEQITENGDVALA
NKQQVTYITERCVFLLKEDGLHLSEIAPGIDLQTQILDVMDFAPI
IDRDANGQIKLMDAALFAEGLMGLKEMKS.

In certain embodiments, the pct gene encodes a CoA-transferase comprising an amino acid sequence having at least 80% identity (e.g., at least 85%, at least 90%, at least 93%, at least 95%, at least 96%, at least 97%, at least 98%, at least 99%, or a 100% identity) to the amino acid sequence set forth in SEQ ID NO:21 and comprising the substitution V193A, where the residue position is with reference to SEQ ID NO:21.

In certain embodiments, the exogenously introduced genes may be integrated into the genome of the microorganism or may be present as an extrachromosomal nucleic acid, e.g., a plasmid.

### Methods

Provided is a method of making a PHA polymer of formula (I):

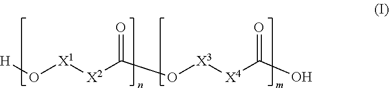

wherein:
n and m define the mol % of each unit within the PHA polymer, wherein n ranges from greater than 0% to 100% and m is 100% minus n.
X¹ and X³ are each independently absent, arylene, heteroarylene, substituted arylene, or substituted heteroarylene; and
X² and X⁴ are each independently alkylene, alkenylene, alkynylene, heterocyclene, arylene, heteroarylene, substituted alkylene, substituted alkenylene, substituted heterocyclene, substituted alkynylene, substituted arylene, or substituted heteroarylene,
the method comprising the step of culturing a microorganism to produce the PHA polymer of formula (I).

As described above, the method is a method of making a PHA polymer of formula (I).

In some cases, X¹ is an absent and X² is alkylene or substituted alkylene. For instance, the “n” monomer can have the structure N1, wherein X¹ is absent and X² is 1,2-diylpropane. This structure can be considered as related to 3-hydroxybutrylate.

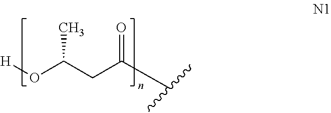

The terms “n” monomer, “n” co-monomer, and “n” unit are used interchangeably herein. Such terms refer to the chemical moiety that is located within the brackets labeled by subscript “n”. Similarly, the terms “m” monomer, “m” co-monomer, and “m” unit are used interchangeably herein to refer to the chemical moiety that is located within the brackets labeled by subscript “m”.

In some embodiments, the “m” monomer is present. In other words, in some cases m is greater than 0%. As described above, m is 100% minus n, and therefore n is less than 100% in the embodiments wherein the “m” monomer is present.

In some cases, X³ is absent and X⁴ is alkylene or substituted alkylene. In some cases, X⁴ is alkylene. In some cases, X⁴ is substituted alkylene, e.g., X⁴ is aryl-alkylene, which is an alkyl group substituted with an aryl group. For instance, if X³ is absent and X⁴ is 2-phenyl-1,1-diylethane, then the “m” monomer will have the structure M1.

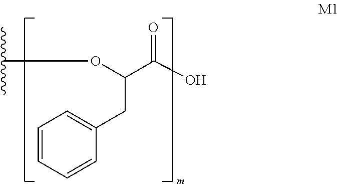

As another example, if X³ is absent and X⁴ is 1-phenyl-1,1-diyl-methane, then the resulting “m” monomer will have the structure M2.

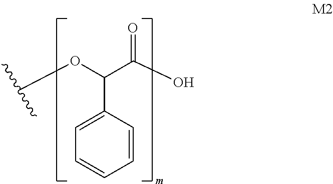

In some embodiments, compounds of formula (I) with such “m” monomers can be generated by adding Compound 1 or 2 to the cell culture medium. For instance, Compound 1 corresponds to “m” monomer M1 wherein X³ is absent and X⁴ is 2-phenyl-1,1-diylethane. Compound 1 is phenylacetate. Similarly, Compound 2 corresponds to an “m” co-monomer M2 wherein X³ is absent and X⁴ is 1-phenyl-1,1-diyl-methane. Compound 2 is mandelate.

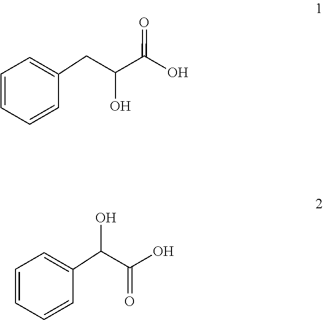 [SMILES: O=C(O)C(O)Cc1ccccc1 ; O=C(O)C(O)c1ccccc1]

In some cases, X³ is arylene or substituted arylene and X⁴ is alkylene or substituted alkylene. For instance, if X³ is phenylene and X⁴ is 1,2-diylethane, then the “m” co-monomer will have structure M3. Such an “m” group can be generated by adding Compound 3 to the cell culture medium. Compound 3 is phloretatic acid.

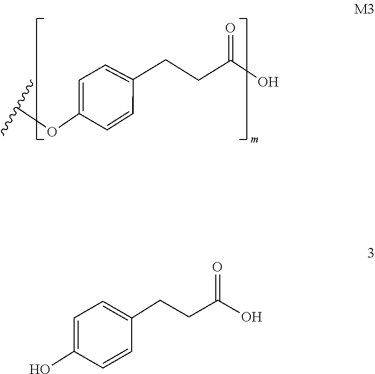

In some instances, X³ is alkylene or substituted alkylene and X⁴ is heteroarylene or substituted heteroarylene. For instance, if X³ is methylene and X⁴ is 2,5-diylfuran, then the “m” co-monomer will have structure M4. Such an “m” group can be generated by adding Compound 4 to the cell culture medium. Compound 4 is 5-hydroxymethyl-2-furancarboxylic acid.

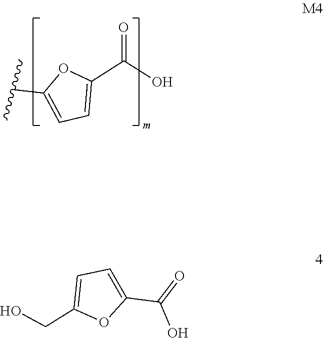

### Compounds

Provided are compounds of formula (I):

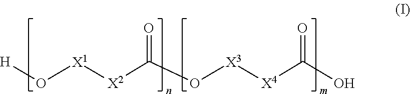

wherein:
n and m define the mol % of each unit within the PHA compound, wherein n ranges from greater than 0% to 100% and m is 100% minus n.
X¹ and X³ are each independently absent, arylene, heteroarylene, substituted arylene, or substituted heteroarylene; and
X⁻ and X⁺ are each independently alkylene, alkenylene, alkynylene, heterocyclene, arylene, heteroarylene, substituted alkylene, substituted alkenylene, substituted heterocyclene, substituted alkynylene, substituted arylene, or substituted heteroarylene.

In some instances, m is greater than 0%, i.e. and thus n is less than 100%. In some instances, n ranges from 5% to 95%, such as from 10% to 90%, from 20% to 80%, or from 30% to 70%. In some n ranges from 5% to 50%, such as from 10% to 40%. In some cases n ranges from 50% to 99%, such as from 55% to 90%.

The compounds of formula (I) can also be referred to as PHA polymers. The term “polymer” as used herein refers to a compound wherein the total number of “n” subunits plus the total number of “m” subunits is 2 or more, such as 5 or more, 10 or more, 25 or more, 50 or more, 100 or more, 1,000 or more, 5,000 or more, or 10,000 or more. In cases wherein both “n” and “m” subunits are present, the polymer can be referred to as a “copolymer” since multiple types of monomers are present. Such monomers can also be referred to as “comonomers” of the copolymer. In some embodiments, the copolymer is an “random copolymer” or “statistical copolymer”, wherein both terms are used interchangeably to refer to copolymers wherein the “n” and “m” subunits are present in a random order. In random copolymers the probability of observing a particular comonomer at a particular position is the mole fraction of the comonomer in the copolymer as a whole. In some cases, the copolymer is an “alternating copolymer” wherein the copolymers alternate, e.g. n-m-n-m-n-m.

In some cases, the “m” monomer has the structure of M3, which can be referred to as a derivative of phloretic acid. In some embodiments, the “n” monomer is N1.

In some cases, the “m” monomer has the structure of M4, which can be referred to as a derivative of 5-hydroxymethyl-2-furancarboxylic acid. In some embodiments, the “n” monomer is N1.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

### Aspects

1. A genetically modified microorganism comprising:
   a heterologous polyhydroxyalkanoate (PHA) synthase (phaC) gene; and
   an isocaprenoyl-CoA:2-hydroxyisocaproate CoA-transferase (hadA) gene and/or a propionate CoA-transferase (pct) gene,
   wherein the microorganism is capable of producing a PHA polymer.
2. The microorganism of claim 1, wherein the species of the microorganism is Cupriavidus necator.
3. The microorganism of claim 2, wherein the Cupriavidus necator is a ΔphaC1 mutant of Cupriavidus necator.
4. The microorganism of any one of claims 1-3, wherein the phaC gene has at least 80% sequence identity to a PHA synthase (phaC) gene from a bacteria of the Pseudomonadaceae genus.
5. The microorganism of claim 4, wherein the bacteria of the Pseudomonadaceae genus is Pseudomonas sp. MBEL 6-19.
6. The microorganism of any one of claims 1-5, wherein the isocaprenoyl-CoA:2-hydroxyisocaproate CoA-transferase (hadA) gene has at least 80% sequence identity to a gene from Clostridium difficile, wherein the microorganism comprises the hadA gene.
7. The microorganism of any one of claims 1-6, wherein the propionate CoA-transferase (pct) gene has at least 80% sequence identity to a gene from Clostridium propionicum, wherein the microorganism comprises the pct gene.
8. The microorganism of any one of claims 1-7, wherein the PHA polymer comprises a carbon atom metabolized from a carbon source by the microorganism.
9. The microorganism of claim 8, wherein the carbon source is selected from the group consisting of: a gaseous mixture comprising CO₂ and H₂, formic acid, acetic acid, fructose, sucrose, or salts thereof.
10. The microorganism of any one of claims 1-9, wherein the PHA polymer has the formula (1):

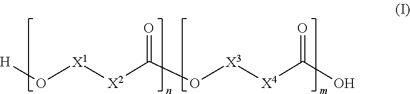

wherein:
n and m define the mol % of each unit within the PHA polymer, wherein n ranges from greater than 0% to 100% and m is 100% minus n.
X¹ and X³ are each independently absent, arylene, heteroarylene, substituted arylene, or substituted heteroarylene; and
X² and X⁴ are each independently alkylene, alkenylene, alkynylene, arylene, heteroarylene, substituted alkylene, substituted alkenylene, substituted alkynylene, substituted arylene, or substituted heteroarylene.
11. A method of making a polyhydroxyalkanoate (PHA) polymer of formula (I):

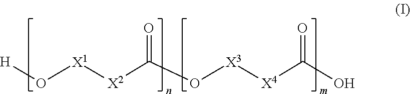

wherein:
n and m define the mol % of each unit within the PHA polymer, wherein n ranges from greater than 0% to 100% and m is 100% minus n.
X¹ and X³ are each independently absent, alkylene, arylene, heteroarylene, substituted arylene, or substituted heteroarylene; and
X² and X⁴ are each independently alkylene, alkenylene, alkynylene, arylene, heteroarylene, substituted alkylene, substituted alkenylene, substituted alkynylene, substituted arylene, or substituted heteroarylene,
the method comprising the step of culturing a microorganism to produce the PHA polymer of formula (I).
12. The method of claim 11, wherein the microorganism is a microorganism according to any one of claims 1-9.
13. The method of any one of claims 11-12, wherein the culturing comprises contacting the microorganism with a carbon source and the PHA polymer comprises a carbon atom from the carbon source.
14. The method of claim 13, wherein the carbon source is selected from the group consisting of: a gaseous mixture comprising CO₂ and H₂, formic acid, acetic acid, fructose, sucrose, and salts thereof.
15. The method of claim 14, wherein the carbon source is a gaseous mixture comprising CO₂ and H₂.
16. The method of any one of claims 11-15, wherein m is greater than 0% and wherein the culturing comprises contacting the microorganism with a compound of formula (II):

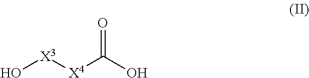

or a salt thereof.
17. The method of claim 16, wherein the compound of formula (II) is selected from the group consisting of:

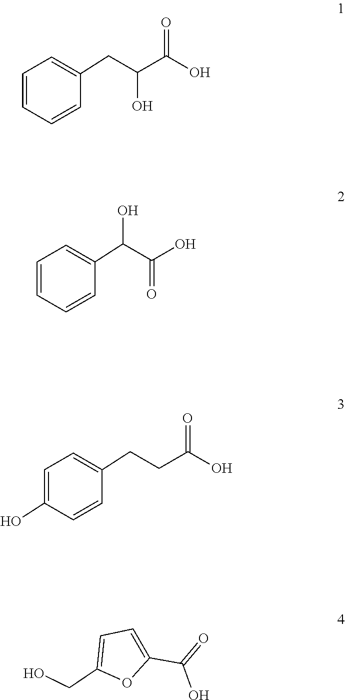 [SMILES: O=C(O)C(O)Cc1ccccc1 ; O=C(O)C(O)c1ccccc1 ; O=C(O)CCc1ccc(O)cc1 ; O=C(O)c1ccc(CO)o1]

and salts thereof.
18. The method of any one of claims 11-17, wherein X¹ is absent and X² is alkylene or substituted alkylene.
19. The method of claim 18, wherein the “n” monomer has the structure of N1:

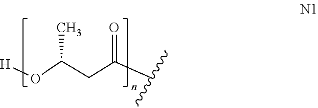

or a stereoisomer thereof.
20. The method of any one of claims 11-19, wherein m is greater than 0% and X³ is absent.
21. The method of claim 20, wherein X⁴ is alkylene or substituted alkylene.
22. The method of claim 21, wherein the “m” monomer has the structure of M1 or M2:

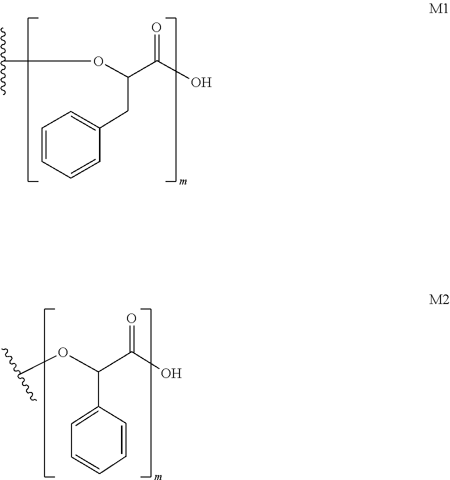

or a stereoisomer thereof.
23. The method of any one of claims 11-19, wherein m is greater than 0% and X³ is arylene or substituted arylene.
24. The method of claim 23, wherein X⁴ is alkylene or substituted alkylene.
25. The method of claim 24, wherein the “m” monomer has the structure of M3:

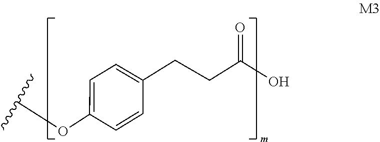

26. The method of any one of claims 11-19, wherein X³ is alkylene and X⁴ is heteroarylene.
27. The method of claim 26, wherein the “m” monomer has the structure of M4:

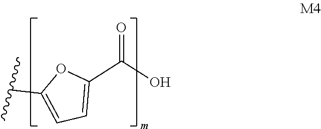

28. A compound of formula (I):

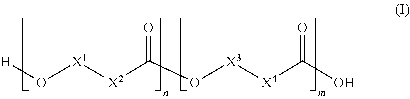

wherein:
n and m define the mol % of each unit within the PHA polymer, wherein n ranges from greater than 0% to 100% and m is 100% minus n.
X¹ and X³ are each independently absent, alkylene, arylene, heteroarylene, substituted arylene, or substituted heteroarylene; and
X² and X⁴ are each independently alkylene, alkenylene, alkynylene, arylene, heteroarylene, substituted alkylene, substituted alkenylene, substituted alkynylene, substituted arylene, or substituted heteroarylene.
29. The compound of clause 28, wherein the “n” monomer has the structure of N1 and the “m” monomer has the structure M3:

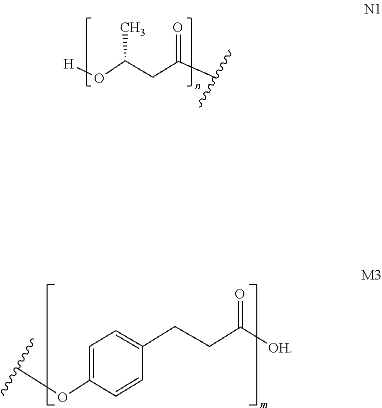

30. The compound of clause 28, wherein the “n” monomer has the structure of N1 and the “m” monomer has the structure M4:

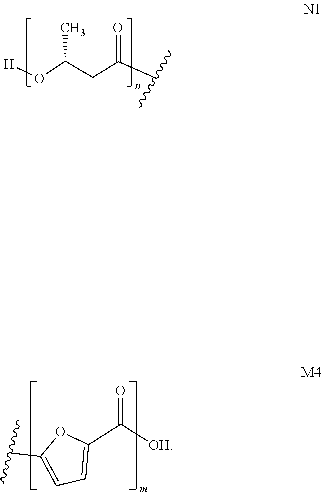

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); and the like.

### Overview

Microbial cell factories have been created that allow the formation of a wide spectrum of aliphatic and aromatic polyesters. Specifically, a ΔphaC1 mutant of the lithoautotroph Cupriavidus necator was complemented with an engineered PHA synthase (phaC1437) from Pseudomonas sp. MBEL 6-19, in combination with a promiscuous isocaprenoyl-CoA:2-hydroxyisocaproate CoA-transferase (hadA) from Clostridium difficile or a mutated propionate CoA-transferase (pct540) from Clostridium propionicum, respectively. Expression of the heterologous genes allowed the incorporation of various non-natural monomers into the polymer: co-polymers of 3 hydroxybutyrate with straight-chain hydroxy carbonic acids like 4 hydroxybutyrate and 6 hydroxycaproate could be obtained. The aromatic hydroxy carbonic acids 3 phenyllactate yielded a co-polymer with an approx. 2:1 ration of 3-hydroxybutyrate to 3 phenyllactate, which significantly altered material properties. Further, we were able to obtain a co-polymer that contained phloretate, for the first time showing incorporation of the aromatic ring in the backbone of a biological polyester. Polymers of phloretic acid have structural analogy with industrial grade high-strength synthetic polyesters and “liquid-crystal” polymers like polyarylates. This opens the door to the bio-production of thermoplastics and thermosets from CO₂, with applications ranging from packaging in food-industry (e.g., PLA and PET) to specialty applications in space-technology (e.g., Vectran™). Synthetic biochemical pathways for de-novo production of the novel PHAs are under development.

### Example 1: Genetic Engineering of Microorganisms

The bacterium employed was a ΔphaC1 mutant of Cupriavidus necator. This bacterium was genetically modified to contain an engineered PHA synthase gene (phaC1437) from a Pseudomonas sp. MBEL 6-19 bacteria. The C. necator bacterium was further genetically modified to include either a hadA gene from Clostridium difficile or a pct540 gene from Clostridium propionicum.

### Materials and Methods

### Vector Construction

The RK2/RP4 oriV (IncP) plasmid pCM66T was obtained from AddGene. pBBR1MCS_P_{BAD}-RFP, a derivate of pBBR1MCS with a red fluorescent protein (RFP) under control of the araBAD promoter, was a gift from the Silver Laboratory (Harvard Medical School). pCM66T_P_{BAD}-RFP was constructed by cloning the P_{BAD}-RFP cassette into pCM66T using NEBuilder, replacing the polylinker and its regulatory elements. The protein sequences of Q9L3F7_CLOPR (pct), Q18813_PEPD6 (hadA) and B9W0T0_9PSED (phaC) were derived from UniProt, implementing the previously described mutations (V193A for pct540 and E130D, S325T, S477G, Q481K for phaC1437) as applicable. The sequences were fused with a C-terminal tripleglycine-spacer and tetracysteine-(Lumio)-tag and codon-optimized for expression in C. necator with GeneArt® (Invitrogen). The genes were arranged in a single operon under control of the araBAD promoter in combination with the strong T7 ribosomal binding-site and a T7Te-rmnBT1 double-terminator. The plasmids pCM66T_P_{BAD}-pct540-phaC1437 and pCM66T_P_{BAD}-hadA-phaC1437 were constructed by GenScript, cloning the synthetic operons containing pc: 540 & phaC1437 and hadA & phaC1437, respectively, into pCM66T_P_{BAD}-RFP.

### Conjugation

Plasmid vectors were introduced into C. necator by conjugation, using the E. coli donor-strain WM3064, which had been transformed with the plasmid vectors pCM66T_P_{BAD}-pct540-phaC1437 and pCM66T_P_{BAD}-hadA-phaC1437 using the Mix & Go! E. coli Transformation Kit (Zymo).

Conjugation was performed as follows: The recipient strain (H16 ΔphaC1) was incubated at 30° C. on RB plates for two days, simultaneously the donor strains carrying the plasmid vectors were incubate at 37° C. on LB+Kan+DAP plates for one day. On the third day the donor strains were inoculate in liquid LB+Kan+DAP and incubated overnight with shaking at 37° C., the recipient strain was inoculated in RB and incubate overnight with shaking at 30° C. On the fourth day 3 mL of LB+DAP (but no antibiotics) were inoculated with 6 μL of each overnight donor-strain culture and incubated with shaking at 37° C. At the same time 20 μL of overnight C. necator culture was added in 10 mL RB and incubated at 30° C. while shaking. After 4 h 3 mL of the E. coli and 10 mL of the C. necator culture were combined and spun down (4816 g for 10 min). The supernatant was discarded, and the cells were re-suspended in 200 μL RB+DAP. A “blob” of the cell mixture was pipetted on an RB+DAP plate and incubated at 30° C. overnight (face of plate up). On day five all of the grown biomass was collected from the overnight plate with an inoculation loop and suspended into 500 μL of 25% glycerol and diluted 1:10 and 1:100. All three concentrations were plated on separate RB+kan plates. The plates were incubated at 30° C., colonies that appeared after 2-3 days were picked and isolated on separate RB+kan plates for screening.

### Example 2: Production of PHA Compounds

The necator bacteria were incubated with cell culture media along with a monomer selected from Compounds 1 (phenylactate), Compound 2 (mandelate), and Compound 3 (phloretate).

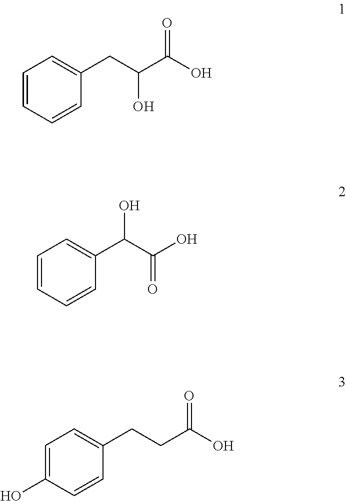 [SMILES: O=C(O)C(O)Cc1ccccc1 ; O=C(O)C(O)c1ccccc1 ; O=C(O)CCc1ccc(O)cc1]

The necator bacteria generated the N1 monomer related to 3-hydroxybutyrate themselves, i.e., without addition of the monomer itself.

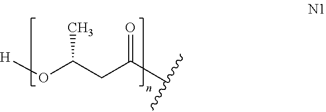

It was observed that the following copolymers were produced, as confirmed by H-NMR. In each case, the copolymers had the N1 monomer. In addition, poly-3HB-co-phenyllactate had the M1 monomer, poly-3HB-co-mandelate had the M2 monomer, and poly-3HB-co-phloretate had the M3 monomer.

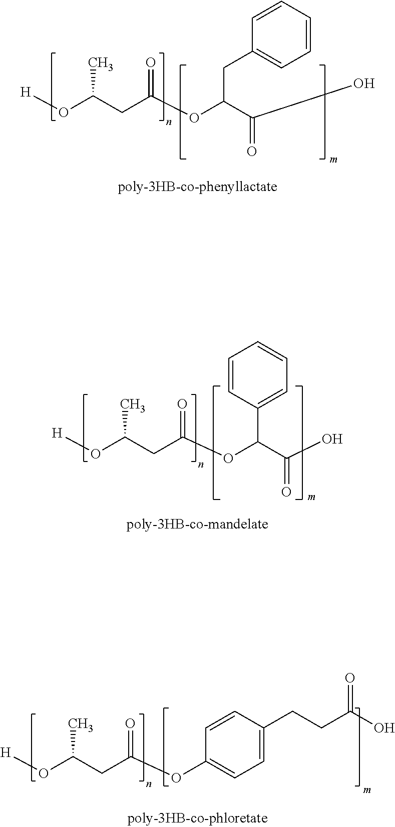

The first co-polymer with phenyllactate had an about 2:1 ratio of 3HB to PheLA. The phloretic acid copolymer product had about a 1:1 ratio of the two comonomers.

### Materials and Methods

Cultivation of C. necator in Shake-and Serum-Flasks

Liquid cultures under heterotrophic conditions were conducted in 500 mL vented, baffled shake-flasks (WHEATON® Erlenmeyer Flasks with DuoCap®. DWK Life Sciences), incubated with shaking at 180 rpm on an innova 2300 platform shaker (New Brunswick Scientific) at 30° C. For feeding experiments, precultures of the engineered C. necator strains were inoculated in liquid RB+kan (50 mL) from solid RB+kan and grown over-night. In the morning the medium was exchanged for MSM+kan, diluting the culture 1:2 (in 100 mL). In the afternoon the seed-culture was washed with MSM and again diluted 1:2 (in 200 mL) with MSM+kan+polymer-precursor and induced with arabinose (1 g/L, unless indicated otherwise) in the evening. OD was monitored accompanied by collection of supernatant samples. The cultures were harvest after approx. 48 h or when no more increase in biomass was observed for 12 h.

Precultures for the bio-electrochemical system were done in two steps: 125 mL serum bottles (25 mL liquid medium, 100 mL gas headspace) were filled with 25 mL electro-medium and sealing with butyl rubber stopper and crimp-cap. Starting with fructose (1 g/L) as carbon-source, the medium was inoculated with C. necator H16 ΔphaC1 pCM66T_P_{BAD}-hadA-phaC1437 from solid RB+kan (heterotrophic growth) and incubated at 30° C. with shaking at 200 rpm overnight. Subsequently, these cultures were transferred to autotrophic conditions on H₂/CO₂/O₂: the initial gas-phase was H₂/CO₂ (80%/20%) at 17 psi, which was further pressurized to 22 psi with O₂ (100%), resulting in a final gas composition of 64:16:20 (H₂/CO₂/O₂). After three days of incubation autotrophic growth was observed, reaching a maximum cell density of OD 3.8±0.15 within 48 h (data not shown). For transfer to the BES exponentially growing cultures were harvested at an OD of ≈0.7 (by centrifugation at 4000×g for 6 min) and re-suspended in 25 mL fresh medium.

Cultivation of C. necator in Bio-electrochemical System

The bio-electrochemical system was a custom (500 mL) glass vessel with rubber-stopper side ports (Adams & Chittenden Scientific). The reactor was operated membrane-less as three-electrode set up, magnetically stirred at 300 rpm. The cathode was a Nickel-Molybdenum alloy on graphite support (total surface area 50 cm²), which has been characterized previously and found to evolve H₂ at 100% selectivity under biologically relevant conditions (F. Kracke et al., Communications Chemistry 2, 45 (2019); F. Kracke, et al., Green Chemistry 22. 6194-6203 (2020)). The anode was platinized titanium mesh (PLANODE1X4, TWL) and an Ag/AgCl reference electrode (NaCl saturated; RE-5B, BASi®), both of which were inserted via a rubber stopper side port. The electrochemical reactor was controlled by applying a constant current of 100 mA using a multichannel potentiostat (VMP3; BioLogic Science Instruments, EC-Lab 11.21). This way, a constant amount of electron flow and therefore constant flow of H₂ and O₂ was provided. In abiotic pre-tests (data not shown) the reactor headspace was analyzed (via GC) to confirm that H₂ and O₂ were the sole gaseous products. The reactor was filled with 300 ml medium, and CO₂ was supplied via a mass flow controller (EL-Flow F-100D, Bronkhorst®) at a constant flow rate of 1 mL/min. Before inoculation, the BES was operated for at least one hour under abiotic conditions to saturate the medium with the gases. The reactor was inoculated with 25 mL of concentrated cell-suspension from exponentially growing, autotrophic cultures, so that a starting OD between 0.5-0.6 (and final liquid volume of 325 mL) was achieved. Preliminary tests showed that under autotrophic growth conditions accumulation of PHA required tight limitation of the nitrogen-source. Therefore, the initial concentration of ammonium salt in the BES was reduced to 5 mM and consumption was monitored (“EasyStrips” Ammonia Test Strips, Tetra® GmbH, sensitivity <0.5 mg/L) throughout the experiment. Growth and pH were also measured by drawing samples manually. After 24 h the culture was induced with arabinose (0.1 g/L final conc.). 24 h after induction, the first dose of precursor was added (2.5 mM final conc.), and the second dose (additional 2.5 mM, i.e., 5 mM total) when the nitrogen-source was depleted. The experiment was terminated when the OD became stationery, cells were harvested via centrifugation followed by polymer extraction.

### Extraction of PHAs

Liquid culture of C. necator was harvested by centrifugation and the cell pellet was freeze dried. After determining the dry cell weight (CDW), PHAs were extracted by lysis of the cell pellet (wet or dry) with 10% sodium hypochlorite solution (Honeywell Fluka™) using approx. 0.2 L/g_{CDW}. The pellet was completely suspended and incubated at room-temperature for 20 min with intermittent mixing. The suspension was diluted with water 1:2 and centrifuged at 4816×g for 20 min. The remaining solids, containing the PHAs, were washed twice with water and once with methanol, repeating the centrifugation step. The dried PHA was weight to determine product yield, dissolved in chloroform, filtered with a 0.2 μm PTFE “Titan3™” (Thermo Scientific™) syringe filter and dried for analysis.

### Protein Extraction and Detection

Culture derived from distinct time-points of a batch cultivation (exponential-phase/stationary-phase) was collected (sample volume [mL]≈10/OD₆₀₀) and cells were pellet by centrifugation (4816×g at 4° C. for 10 min). The pellet was washed with purified water and stored as cell-paste at −20° C. for later processing. For extraction of proteins, CelLytic™ B (Sigma) was used as per manufacturer's directions (approx. 1 mL per cells from 10 mL culture at an OD of 1), in combination with Protease Inhibitor Cocktail (Sigma-Aldrich). The mixture was vortexed for 2 min to lyse cells and extract the soluble protein. Centrifugation (4816×g for 10 min) pelleted the cell debris; the supernatant, which contained the soluble proteins, was separated. Total protein concentration was determined using the BCA Protein Assay Kit (Pierce™). Using the Lumio™ Green Detection Kit (ThermoFisher) as per manufacturer's directions 10 μg crude protein extract of each sample were prepared for gel electrophoresis. Size-separation was performed on a Bolt™ 4-12% Bis-Tris Plus Gel (ThermoFisher), run at 150 V for approx. 40 min with Bolt™ MES SDS Running Buffer (ThermoFisher). The marker was BenchMark™ Fluorescent Protein Standard (ThermoFisher). A Gel-Doc (BioRad) was used to visualize fluorescent-conjugated proteins. For visualization of all proteins, the gels were re-stained with One-Step Lumitein™ Protein Gel Stain (Biotium) as per manufacturer's directions and imaged again as before.

### Analytics

### Determination of OD and Cell Dry Weight Correlation

Microbial growth was characterized by measuring the optical density at 600 nm (OD₆₀₀) with a DR2800™ Portable Spectrophotometer (HACH) for shake-flask cultures and Ultrospec™ 2100 pro (Amersham BioSciences) in case of MES.

A correlation between OD₆₀₀ and biomass (BM) concentration was determined gravimetrically (data not shown) from batch shake-flask cultivations with the wild-type (five samples) and engineered strains (five samples of the pct540-strain, 10 samples of the hadA-strain) of C. necator. Shake-flask cultures of 50 mL with different cell densities were harvested via centrifugation and vacuum dried. The average quotient of OD₆₀₀/BM (dry weight in mg) from the total of 20 samples was 0.3±0.04, such that the correlation is OD₆₀₀×0.3=BM [g/L].

### HPLC

Quantification of fructose in fermentation broth was based on a previously published HPLC-method for detection of organic acids (S. T. Lohner, et al. The ISME Journal 8, 1673-1681 (2014)). In short, the procedure was as follows: Samples (1 mL) were filtered (PVDF or PES syringe filters, 0.2 μm pore-size) and diluted 1:100 into HPLC sampling vials. Analysis of 50 μL sample-volume was performed on an 1260 Infinity HPLC system (Agilent), using an Aminex HPX87H column (BioRad) with 5 mM H₂SO₄ as the eluent, at a flow rate of 0.7 mL/min. Fructose was identified and quantified by comparison to standards (3 g/L, 1.5 g/L, 0.6 g/L, 0.3 g/L, 0.15 g/L, 0.03 g/L), according to retention time (8.8 min) using a refractive index detector (35° C.).

### Nuclear Magnetic Resonance (NMR) Spectroscopy

NMR samples were prepared as previously reported (J. Myung et al., Bioresource Technology 198, 811-818 (2015)). In short, a few mg of polymer were dissolved in deuterated chloroform and ¹H-NMR as well as ¹³C-NMR spectra were recorded at 25° C. on a Unity INOVA™ 500 NMR Spectrometer (Varian Medical Systems) with chemical shifts referenced in ppm relative to tetramethylsilane.

### Gas Chromatography-Mass Spectrometry (GC-MS)

GC-MS analysis was adopted from literature (S. N. Nangle et al., Metabolic Engineering 62, 207-220 (2020)). Between 3-70 mg of extracted PHAs were transferred to crimped vials and 2 mL chloroform+2 mL methanol with 15% HCl was added. The vials were closed and incubated for 1-2 h at 100° C. Vials were cooled on ice and content was combined with 4 mL H₂O in a screw cap glass vial. The mixture was vortexed and phases were separated by centrifugation at 3000 rpm for 10 min. The lower chloroform phase was transferred into a GC-MS vial for analysis. Samples were analyzed on a 7890/5975 inert XL GCMS (Agilent Technologies) with a J&W CP-TAP CB column CP7483 (Agilent Technologies). Analytes were heated on a gradient from 35-250° C. at 2° C./min. Copolymers were detected with mass spectra of hydroxy acid methyl esters at m/z=1-3 and NIST Mass Spectral Library.

### Gel Permeation Chromatography (GPC)

Polystyrene calibrated (from Mₚ=500-275,000 g/mol) molecular weights were determined using a GPCmax autosampler (Viscotek) with 300 mm×7.7 mm GPC column (Waters™) in CHCl₃ at 25° C. at a flow rate of 1 mL/min and S3580 refractive index detector (Viscotek).

### Metabolic Modelling

Based on previously established metabolic networks of C. necator for elementary flux-mode analysis (N. J. H. Averesch, F. Kracke. Frontiers in Energy Research 6 (2018), P. Unrean, et al. Bioresources and Bioprocessing 6, 49 (2019)), the present model was fundamentally re-constructed, refined and fully compartmentalized. Expansions were made to describe C1- and energy-metabolism more precisely (N. J. Claassens et al., Proceedings of the National Academy of Sciences 117, 22452-22461 (2020)., R. Cramm, Microbial Physiology 16, 38-52 (2009) and the model was amended with additional carbon assimilation and product formation pathways, deducted from metabolic databases such as KEGG. Reaction thermodynamics of the heterologous pathways were verified with eQuilibrator.

Elementary flux modes were calculated in MATLAB® (MathWorks®), using ‘FluxModeCalculator’, and evaluated as described previously. Balances were established around boundary reactions, allowing carbon-yields [C-mol/C-mol] for all products to be determined.

### Example 3: Influence of Carbon Source on Yield

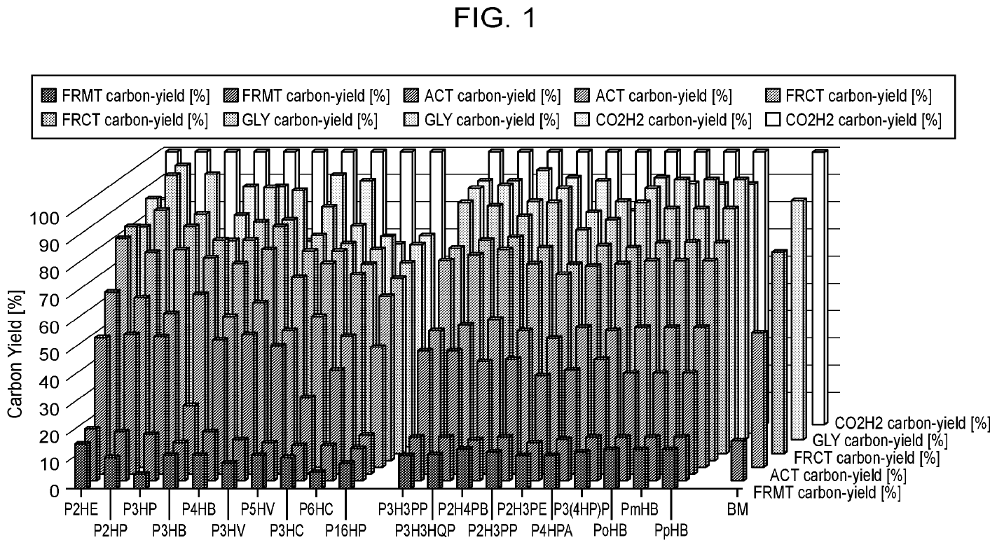

The incubation procedure was modified to provide the necator bacteria with different carbon sources, e.g. for the generation of the 3HB comonomer. As shown in FIG. 1, yields depended on the source. Bacteria fed with CO₂ and H₂ had the highest yield of about 100%, whereas glucose had the next highest yield, followed by fructose, followed by acetate, followed by formate.

### Example 4: Mechanism of Production

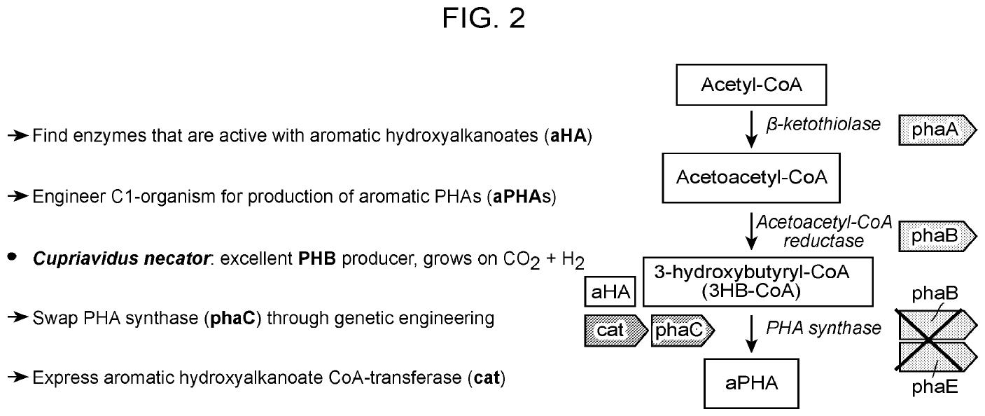

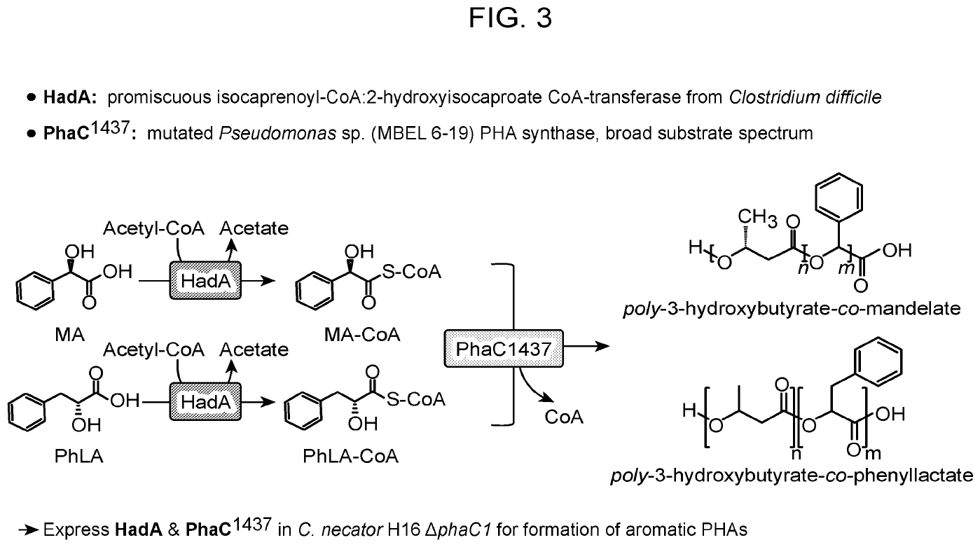

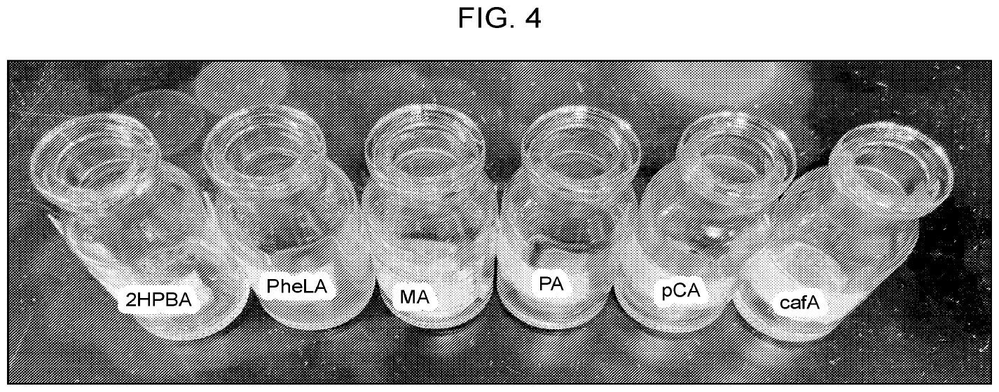

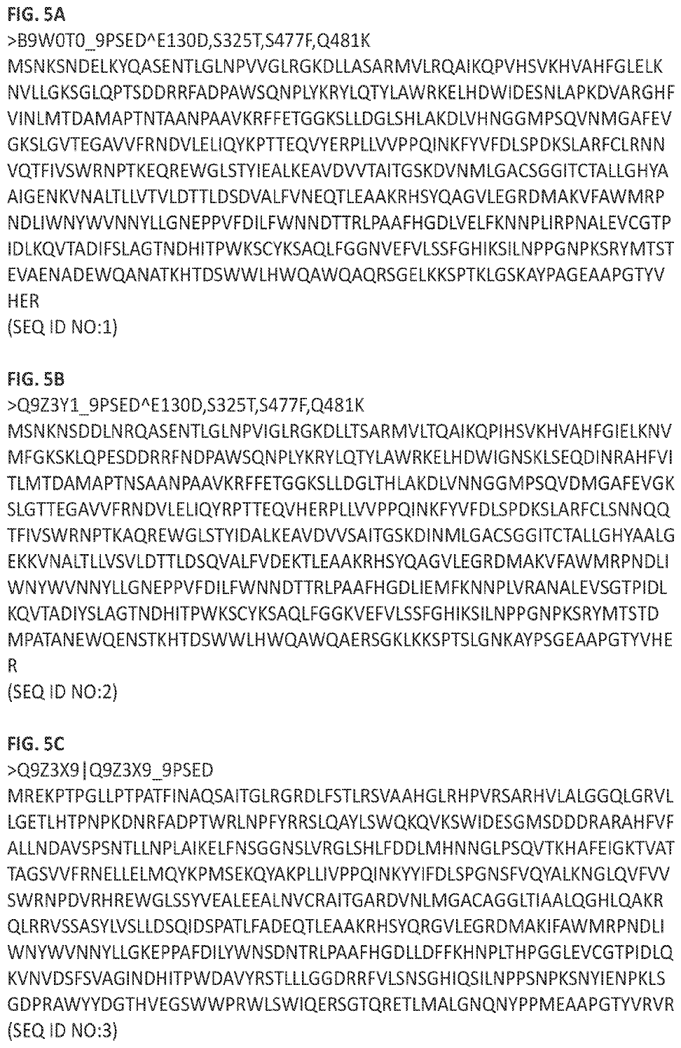

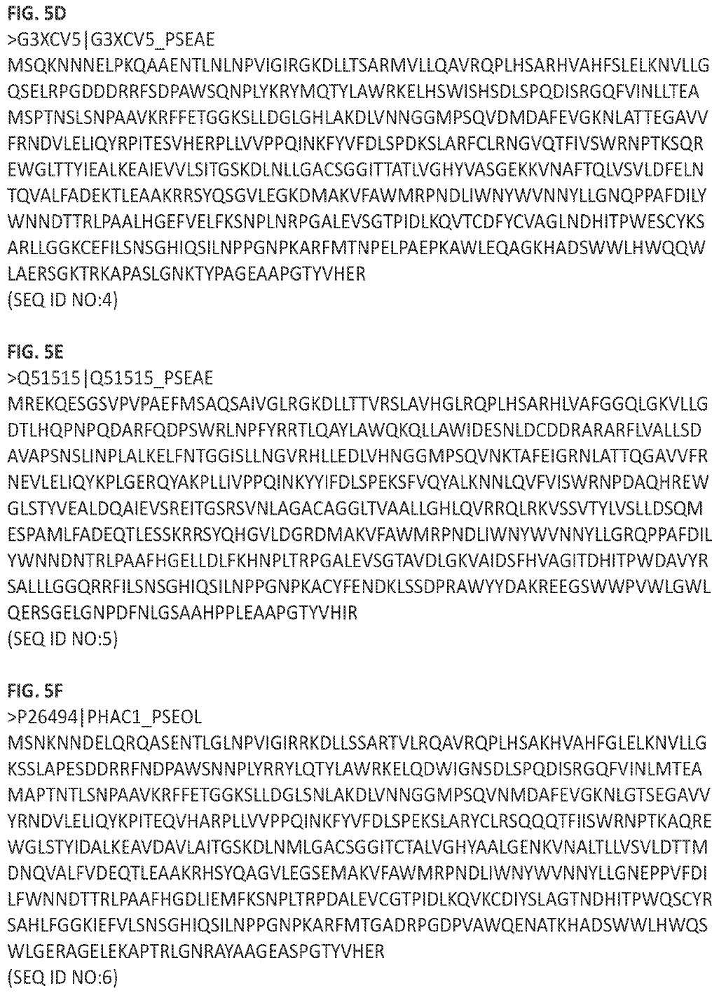

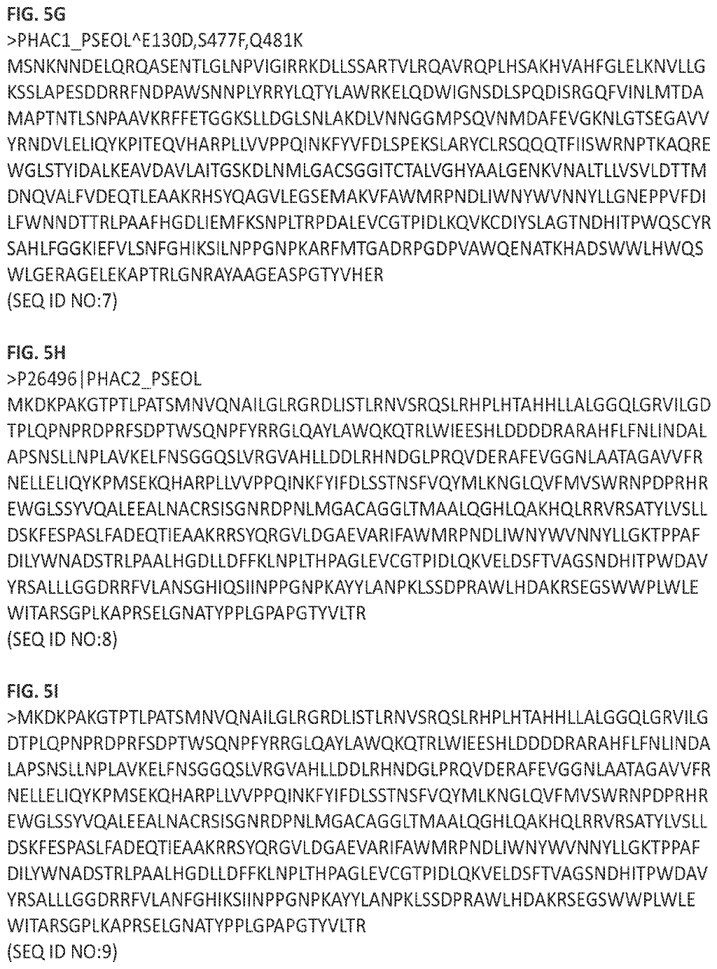

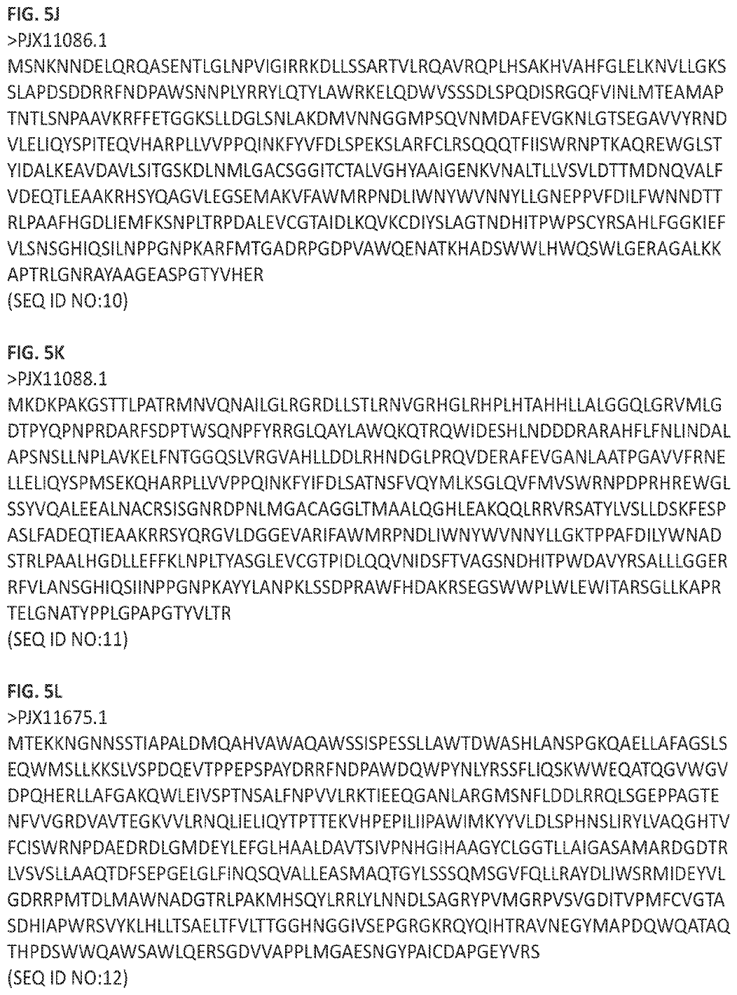

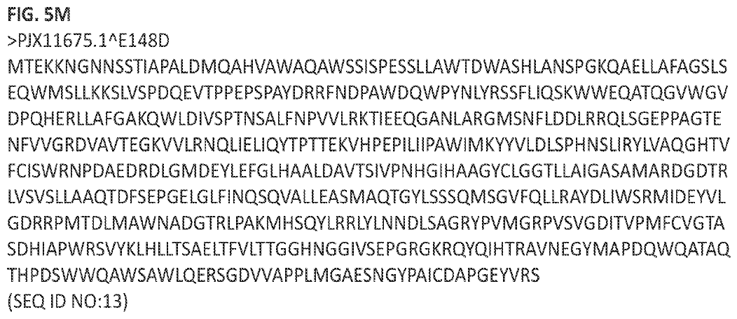

A hypothesized mechanism for the production of PHA compounds by the bacteria is shown in FIGS. 2 and 3.

### Example 5: Additional Studies

Results from additional studies are shown in the table below.

| | | | final | PHA |
|---|---|---|---|---|
| | | Concentration | biomass | yield |
| Strain | Hydroxyalkanoate | [mM] | [g/L] | [w/w] |
| H16 wild-type | none | N/A | n.d. | 20.3 |
| | glycolic acid | 20 | 6.8 | 78.6 |
| | lactic acid | 20 | 18.2 | 24.6 |
| | 3-hydroxypropanoic | 20 | 5.7 | 73 |
| | acid | | | |
| | 4-hydroxybutanoic | 20 | 4.1 | 68.7 |
| | acid | | | |
| | 6-hydroxyhexanoic | saturated | 3.5 | 73.6 |
| | acid | (<10 mM) | | |
| H16 ΔphaC1 | none | N/A | n.d. | n.d. |
| pct540-phaC1437 | glycolic acid | 20 | 1.4 | 07.2 |
| | lactic acid | 20 | 1.9 | 32.3 |
| | 3-hydroxypropanoic | 20 | 1.6 | 29.6 |
| | acid | | | |
| | 4-hydroxybutanoic | 20 | 1.9 | 37.4 |
| | acid | | | |
| | 6-hydroxyhexanoic | saturated | 1.5 | 31.6 |
| | acid | (<10 mM) | | |
| H16 ΔphaC1 | none | N/A | n.d. | 61.8 |
| hadA-phaC1437 | glycolic acid | 20 | 4.3 | 68.1 |
| | lactic acid | 20 | 4.7 | 69.4 |
| | 3-hydroxypropanoic | 20 | 4.1 | 68 |
| | acid | | | |
| | 4-hydroxybutanoic | 20 | 6.1 | 39.3 |
| | acid | | | |
| | 6-hydroxyhexanoic | saturated | 2.7 | 57.2 |
| | acid | (<10 mM) | | |
| | 2-hydroxy-4- | 15 | 0.4 | 47.6 |
| | phenylbutanoic acid | | | |
| | phenyllactic acid | 10 | 0.9 | 42.2 |
| | mandelic acid | 5 | 1.8 | 63.1 |
| | phloretic acid | 5 | 1.4 | 46.7 |
| | para-coumaric acid | 5 | 1.1 | 46.8 |
| | caffeic acid | 5 | 1.1 | 45.5 |

This work focused on characterization of a system for production of non-natural PHAs based on the mixotrophic gas-fermenting betaproteobacterium Cupriavidus necator. In a PHA-negative knock-out mutant (ΔphaC1) of the type-strain ‘H16’, pct540 & phaC1437 and hadA & phaC1437 were expressed, respectively. Formation of PHAs was demonstrated by cultivating the organism under preferred conditions (fructose as main carbon-source for proof-of-concept) and external supply of different aliphatic and aromatic hydroxy carbonic acids at highest non-toxic concentrations (cf. table 1), to maximize incorporation into the poly (3-hydroxybutyrate) (P3HB) co-polymer.

Toxicity limits of the respective hydroxy carbonic acids were determined outgoing from 20 mM down in steps of 5 mM until acceptable growth was obtained in shake-flask experiments at 30° C. Further parameters of the cultivations, as well as results pertaining biomass yield are given as part of table 1.

Table 1: Strains tested for production of PHAs with different hydroxy carbonic acids (maximum non-toxic concentrations). Cultivations conducted on minimal salt medium with 30 g/L fructose as carbon-source and 2 g/L ammonium chloride as nitrogen-source (limiting). Cultures were inoculated at an OD600 of ≈0.5-1.

The average PHA yield of the wild-type was 64% [w/w], while for the variants carrying pct540 only around 28% [w/w] were obtained. With 57% [w/w] the hadA strain almost reached the product yield of the wild-type. This indicates higher activity of HadA in comparison to Pct540 for thioester formation. The high biomass yields in case of cultivations with lactic and glycolic acid could indicate co-utilization of these compounds as carbon-source, which is highly likely, considering C. necator's mixotrophic nature and growth on volatile fatty acids. Lower final biomass in case of the cultivations with aromatic hydroxy carbonic acids may be attributable to their toxicity. The growth-limiting (toxic) effect was more pronounced the shorter the sidechain/the closer the aromatic ring was located in respect to the molecule's backbone; or put the other way round, the closer the functional groups were to the aromatic ring. A qualitative difference of the produced polymer could already be observed in comparison to P3HB produced by the wild-type: PHA from the engineered strains appeared more brittle, indicating a low molecular weight. However, most striking was the observation that polymer obtained from cultures with phloretic and para-coumaric acid were yellow (FIG. 4).

NMR spectroscopy revealed that for the transgenic strains cultivated without additional hydroxy carbonic acids glycolic, lactic and 3-hydroxypropionic acid the product was mostly composed of P3HB (indicated by a sextet peak at 5.26 ppm). In the case of the strain carrying the hadA gene, however, the spectrum differed slightly (additional triplet peak at 4.98 ppm) indicating a co-polymer with 2-hydroxybutryate (2HB). For 4-hydroxybutyric acid (4HB) incorporation into the polymer could be confirmed, identifiable by the peak at 4.11 ppm (FIG. 3). 4HB content was high in case of the strain carrying the pct540 gene: from the peak areas a 4HB fraction of >50% could be estimated. This is considerably more than for the wild-type, which also appeared to be able to incorporate 4HB into the polymer to a small degree (<10%). Further, it appeared that 6-hydroxycaproic acid (6HC) was metabolized into P4HB, likely through β-oxidation, as both strains fed with 6HC showed a P3HB-P4HB co-polymer. Only in case of the strain carrying the hadA gene of 6HC incorporation was observed, which is likely due to the higher activity of the CoA-transferase, being able to compete with catabolismof the monomer. Surprisingly, incorporation of 4HB into the co-polymer was low (≈10%) for the HadA strain fed with 4HB. In light of the much higher amount of total PHA produced by these strains, this suggests that 3HB outcompeted the other monomer(s) rather than the conclusion that HadA has lower affinity for 4HB. In reverse, this does, however, suggest lower affinity of the Pct540 for 3HB.

Incorporation of aromatic monomers into PHAs by strain ‘ΔphaC1 hadA-phaC1437’ has been confirmed for the first time. Peaks indicating the presence of aromatics were found for cultures fed with 2-hydroxy-4-phenylbutanoic, phenyllactic, mandelic and phloretic acid. C¹³-NMR analysis of 2-hydroxy-4-phenylbutanoic, mandelic, and phloretic acid are underway to determine polymer composition, incorporation of a high fraction (≈50%) of phenyllactic acid into the polymer could be confirmed by ¹H-NMR.

### Example 5: Melting Point, Glass Transition Temperature, and Thermal Decomposition

Samples 1-8 were generated according to the methods described in the table below.

| Sample | Strain | HA | Induced | Precursor | Polymer | Ratio |
|---|---|---|---|---|---|---|
| 1 | H16 wild-type | — | #N/A | none | P3HB | #N/A |
| 2 | H16 ΔphaC pct540- | — | no | none | P3HB | #N/A |
| | phaC1437 | | | | | |
| 3 | | — | yes | none | P3HB | #N/A |
| 4 | H16 ΔphaC hadA- | — | no | none | P3HB | #N/A |
| | phaC1437 | | | | | |
| 5 | | — | yes | none | P3HB | #N/A |
| 6 | H16 ΔphaC pct540- | 4HBA | yes | 4-hydroxybutyrate | P(3HB:4HB) | 1 to 1 |
| | phaC1437 | | | | | |
| 7 | H16 ΔphaC hadA- | 6HCA | yes | 6-hydroxyhexanoate | P(3HB:4HB:6HC) | 100 to 0.5 |
| | phaC1437 | | | | | to 0.5 |
| 8 | | PheLA | yes | phenyllactate | P(3HB:PheLA) | 100 to 25 |

The glass transition temperature (T_{g}) in ° C. along with the melting temperature (Tₘ) in ° C. was recorded. Furthermore, it was assessed whether or not the samples were crystalline.

Differential scanning calorimetry (DSC) was conducted on TA Instrument's Q2500 Differential Scanning calorimeter. ˜5 mg samples were prepared in TA instruments standard aluminium “Tzero” pans. Heat flow (mW) was recorded relative to an empty reference pan. Experiments were conducted under 50 mL/min dry N2 flow with a temperature ramp of 10° C./min. Glass transition temperatures (Tg) were determined by taking the midpoint of the transition curve during the second heating-cycle.

Crystallinity is seen in the presence and size of an exothermic peak at a lower temperature than the melting point. Percent crystallinity was determined by the ratio of areas of the crystallization peak and the sum of areas of crystallization and melting peaks.

| Sample | T_{g} (C.) | Tₘ (° C.) | crystalline |
|---|---|---|---|
| 1 | 4 | 170 | yes |
| 2 | 4 | 170 | yes |
| 3 | −6.2 | 143 | yes |
| 4 | 3.9 | 169 | yes |
| 5 | 2.8 | 163 | yes |
| 6 | −25 | 160 | yes |
| 7 | −4.8 | 129 | yes |
| 8 | 11.7 | N/A | no |

These results indicate that Samples 1 and 2 had the highest melting point, along with the largest difference between glass transition and melting point. Hence, Samples 1 and 2 could be expected to have the widest range of processable temperatures, e.g., based on a large temperature range at which they are malleable. Furthermore, quantitative crystallinity (e.g. as a percentage) can be compared between samples. In some instances, a low crystallinity (e.g. a low percentage of crystallinity) in combination with a low glass transition temperature (Tg) could correspond to a more elastomeric polymers, which can be advantageous for changing the shape of the materials. The elastomeric properties of the materials could be quantified with stress-strain data.

In addition, tests with poly-3HB-co-phenylactate showed that it had a decomposition temperature of above 250° C., giving it a large temperature range where it could be malleable enough to be processed with low amounts of decomposition.

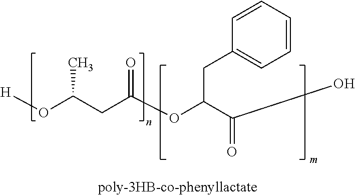

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims. The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A genetically modified microorganism comprising:
a heterologous polyhydroxyalkanoate (PHA) synthase (phaC) gene; and
an isocaprenoyl-CoA:2-hydroxyisocaproate CoA-transferase (hadA) gene and/or a propionate CoA-transferase (pct) gene,
wherein the microorganism is capable of producing a PHA polymer.

2. The microorganism of claim 1, wherein the species of the microorganism is Cupriavidus necator.

3. The microorganism of claim 2, wherein the Cupriavidus necator is a ΔphaC1 mutant of Cupriavidus necator.

4. The microorganism of claim 1, wherein the phaC gene has at least 80% sequence identity to a PHA synthase (phaC) gene from a bacterium of the Pseudomonadaceae genus.

5. The microorganism of claim 4, wherein the bacterium of the Pseudomonadaceae genus is Pseudomonas sp. MBEL 6-19.

6. The microorganism of claim 1, wherein the isocaprenoyl-CoA:2-hydroxyisocaproate CoA-transferase (hadA) gene has at least 80% sequence identity to a gene from Clostridium difficile, wherein the microorganism comprises the hadA gene.

7. The microorganism of claim 1, wherein the propionate CoA-transferase (pct) gene has at least 80% sequence identity to a gene from Clostridium propionicum, wherein the microorganism comprises the pct gene.

8. The microorganism of claim 1, wherein the PHA polymer comprises a carbon atom metabolized from a carbon source by the microorganism.

9. The microorganism of claim 8, wherein the carbon source is selected from the group consisting of: a gaseous mixture comprising CO₂ and H₂, formic acid, acetic acid, fructose, sucrose, or salts thereof.

10. The microorganism of claim 1, wherein the PHA polymer has the formula (I): 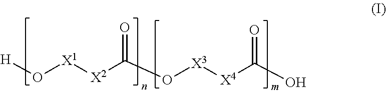
wherein:
n and m define the mol % of each unit within the PHA polymer, wherein n ranges from greater than 0% to 100% and m is 100% minus n.
X¹ and X³ are each independently absent, arylene, heteroarylene, substituted arylene, or substituted heteroarylene; and
X² and X⁴ are each independently alkylene, alkenylene, alkynylene, arylene, heteroarylene, substituted alkylene, substituted alkenylene, substituted alkynylene, substituted arylene, or substituted heteroarylene.

11. A method of making a polyhydroxyalkanoate (PHA) polymer of formula (I): 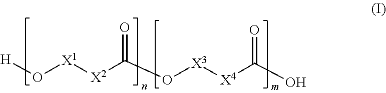
wherein:
n and m define the mol % of each unit within the PHA polymer, wherein n ranges from greater than 0% to 100% and m is 100% minus n.
X¹ and X³ are each independently absent, alkylene, arylene, heteroarylene, substituted arylene, or substituted heteroarylene; and
X² and X⁴ are each independently alkylene, alkenylene, alkynylene, arylene, heteroarylene, substituted alkylene, substituted alkenylene, substituted alkynylene, substituted arylene, or substituted heteroarylene,
the method comprising the step of culturing a microorganism to produce the PHA polymer of formula (I).

12. The method of claim 11, wherein the microorganism is a microorganism according to claim 1.

13. The method of claim 11, wherein the culturing comprises contacting the microorganism with a carbon source and the PHA polymer comprises a carbon atom from the carbon source.

14. The method of claim 13, wherein the carbon source is selected from the group consisting of: a gaseous mixture comprising CO₂ and H₂, formic acid, acetic acid, fructose, sucrose, and salts thereof.

15. The method of claim 14, wherein the carbon source is a gaseous mixture comprising CO₂ and H₂.

16. The method of claim 11, wherein m is greater than 0% and wherein the culturing comprises contacting the microorganism with a compound of formula (II): 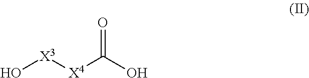
or a salt thereof.

17. The method of claim 16, wherein the compound of formula (II) is selected from the group consisting of: 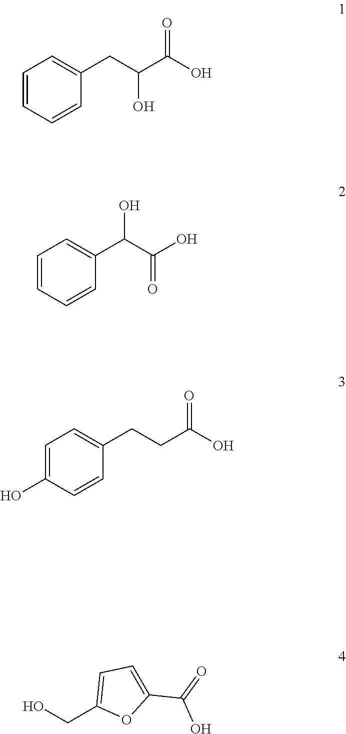 [SMILES: O=C(O)C(O)Cc1ccccc1 ; O=C(O)C(O)c1ccccc1 ; O=C(O)CCc1ccc(O)cc1 ; O=C(O)c1ccc(CO)o1]
and salts thereof.

18. The method of claim 11, wherein X¹ is absent and X² is alkylene or substituted alkylene.

19. The method of claim 18, wherein the “n” monomer has the structure of N1: 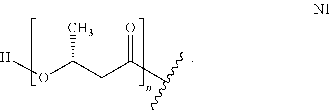
or a stereoisomer thereof.

20. The method of claim 11, wherein m is greater than 0% and X³ is absent.

21. The method of claim 20, wherein X⁴ is alkylene or substituted alkylene.

22. The method of claim 21, wherein the “m” monomer has the structure of M1 or M2: 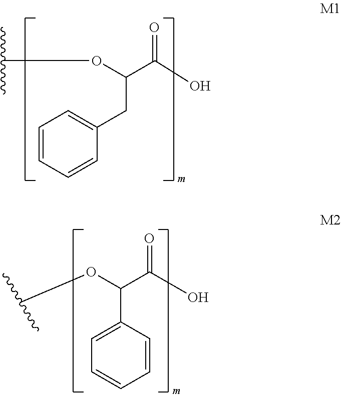
or a stereoisomer thereof.

23. The method of claim 11, wherein m is greater than 0% and X³ is arylene or substituted arylene.

24. The method of claim 23, wherein X⁴ is alkylene or substituted alkylene.

25. The method of claim 24, wherein the “m” monomer has the structure of M3:

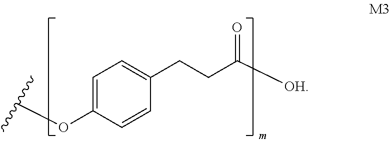

26. The method of claim 11, wherein X³ is alkylene and X⁴ is heteroarylene.

27. The method of claim 26, wherein the “m” monomer has the structure of M4:

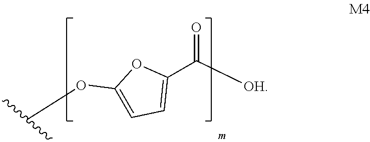

28. A compound of formula (I): 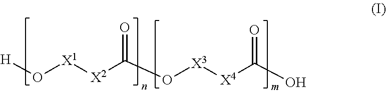
wherein:
n and m define the mol % of each unit within the PHA polymer, wherein n ranges from greater than 0% to 100% and m is 100% minus n.
X¹ and X³ are each independently absent, alkylene, arylene, heteroarylene, substituted arylene, or substituted heteroarylene; and
X² and X⁴ are each independently alkylene, alkenylene, alkynylene, arylene, heteroarylene, substituted alkylene, substituted alkenylene, substituted alkynylene, substituted arylene, or substituted heteroarylene.

29. The compound of claim 28, wherein the “n” monomer has the structure of N1 and the “m” monomer has the structure M3:

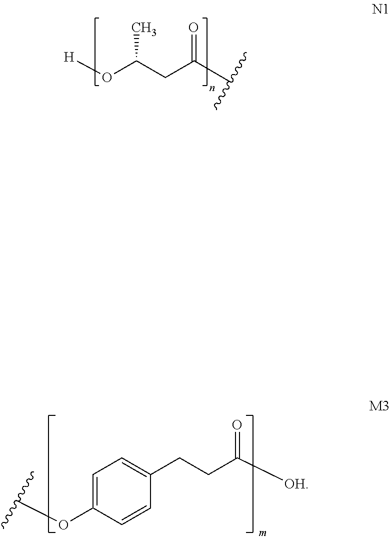

30. The compound of claim 28, wherein the “n” monomer has the structure of N1 and the “m” monomer has the structure M4: [chemical structure]
